(19) United States
(12) Patent Application Publication
(10) Pub. No.: US 2021/0220482 A1
(43) Pub. Date: Jul. 22, 2021

(54) MITOCHONDRIAL COPPER DEPLETION REPROGRAMS THE METABOLISM OF TRIPLE NEGATIVE BREAST CANCER

(71) Applicant: The Board of Trustees of the Leland Stanford Junior University, Stanford, CA (US)

(72) Inventors: Jianghong Rao, Palo Alto, CA (US); Liyang Cui, Sunnyvale, CA (US)

(21) Appl. No.: 17/116,357

(22) Filed: Dec. 9, 2020

Related U.S. Application Data
(60) Provisional application No. 62/945,275, filed on Dec. 9, 2019.

(51) Int. Cl. A61K 47/69; A61K 9/51; A61P 35/00; A61K 49/22; A61K 31/4439

(52) U.S. Cl. CPC A61K 47/6935; A61K 9/5146; A61K 31/4439; A61K 49/222; A61P 35/00

(57) ABSTRACT

Provided is a mitochondrial copper depleting strategy that exploits the potential vulnerability for this metabolic by cancer cells such as Triple Negative Breast Cancer cells. A nanoparticle is provided that comprises a self-reporting copper-depleting moiety (CDM) embedded in or on the matrix comprising a semi-conducting polymer and a phospholipid-polyethylene glycol (PEG). The positively charged copper-depleting complex targets mitochondria and deprives cytochrome c oxidase of its necessary copper co-factor. Inhibition of the electron transport chain complex IV compromises oxygen consumption and abrogates fatty acid oxidation, resulting in energy deficiency induced apoptosis of the targeted cancer cells. The copper-depleting nanoparticle can report the copper depleting status through multimodal optical signal changes while decreasing the copper level in tumors to inhibit tumor growth with low toxicity and significantly prolonged survival.

### CROSS-REFERENCE TO RELATED APPLICATIONS

This application claims priority to U.S. Provisional Patent Application Ser. No. 62/945,275 entitled “MITOCHONDRIAL COPPER DEPLETION REPROGRAMS THE METABOLISM OF TRIPLE NEGATIVE BREAST CANCER” filed on Dec. 9, 2019, the entirety of which is hereby incorporated by reference.

### STATEMENT ON FUNDING PROVIDED BY THE U.S. GOVERNMENT

This invention was made with Government support under contract W81XWH1810591 awarded by the Department of Defense and under contracts CA196585, CA197713, CA243033, and NS069375 awarded by the National Institutes of Health. The Government has certain rights in the invention.

### TECHNICAL FIELD

The present disclosure is generally related to copper-chelating composition. The present disclosure is also generally related to use of the copper-chelating composition for the detection and treatment of a tumor.

### BACKGROUND

Breast cancer is among the deadliest cancers in women ((American Cancer Society. (2018) Cancer Facts Figs. 2018; Howlader et al., (2015) Cancer Statistics Rev. 1975-2014—SEER statistics). An estimated 90% of breast cancer deaths are due to metastasis, with the compelling fact that there is no cure once the metastasis occurs (Ferlay et al., (2015) Int. J. Cancer 136: E359-E386). Among the four major molecular subtypes of breast cancer, the phenotype of ER-/PR-/HER2-, often called triple-negative breast cancer (TNBC), is notoriously known for its high metastatic risk and low overall survival (Foulkes et al., (2010) N. Engl. J. Med. 363: 1938-1948). TNBC is not responsive to endocrine therapy or HER2-targeted therapies. Current TNBC treatment often combines surgery, radiation therapy and chemotherapy, but still yields the poorest prognosis outcome (Bianchini et al., (2016) Nat. Rev. Clin. Oncol. 13: 674-690). To date, only Lynparza has been approved by FDA as targeted therapy for TNBC with BRCA mutations, accounting for less than 20% of the TNBC patients. TNBC is an intrinsically heterogeneous disease. Therefore, targeting a single biomarker or oncogene often yields unsatisfactory therapeutic outcomes. New approaches must be taken to improve therapeutic benefits.

TNBC cells have a metabolic preference for importing and utilizing lipids as energy source. This is likely due to the proximity of primary breast tumor to the adipose-rich mammary gland. Fatty acid oxidation is also found essential in the activation of the Src pathway in TNBC cells (Park et al., (2016) Cell Rep. 14: 2154-2165). Pharmacologic inhibition of fatty acid oxidation blocked the tumor growth and metastasis in transgenic TNBC models (Camarda et al., (2016) Nat. Med. 22: 427-432; van Weverwijk et al., (2019) Nat. Commun. 10: 2698). Moreover, it has been shown that TNBC shows less sensitivity to enzyme inhibitors that target the glucose transport than receptor-positive breast cancer types that rely more on glycolysis (Srirangam et al., (2006) Clin. Cancer Res. 12: 1883-1896; Koppenol et al., (2011) Nat. Rev. Cancer 11: 325-337). Metabolically, glycolysis-dependent cancer types have been shown to induce fatty acid synthesis, which in turn inhibits fatty acid oxidation (Gouw et al., (2019) Cell Metab. 30: 556-572). Therefore, it is attractive to target oxidative phosphorylation (OXPHOS) and inhibit fatty acid oxidation for the treatment of TNBC.

Data mining studies of the breast cancer patients showed evident upregulation in the mitochondrial copper chaperone and co-chaperone proteins COX17 and SCO2 (Nagaraja et al., (2006) Oncogene 25: 2328-2338; Blockhuys et al., (2017) Metallomics 9: 112-123), suggesting breast cancer cells have a higher demand for copper trafficking to the mitochondria than normal cells do. Mitochondrial copper enzyme, cytochrome c oxidase (COX), is the complex IV of the electron transport chain and responsible for energy generation and mitochondrial electrochemical gradient maintenance (Hargreaves et al., (2007) Mitochondrion 7: 284-287; Li et al., (2007) J. Biol. Chem. 282: 17557-17562). COX is matured in the mitochondrial intermembrane space and its activity is subject to the copper supply in the mitochondria (Carr & Winge (2003) Acc. Chem. Res. 36: 309-316). Therefore, limiting the availability of copper will impact the metabolism of cancer cells, especially those in high demand for copper. Indeed, inhibition of copper trafficking with chaperone inhibitors for Atox 1 and CCS has been shown to disrupt bioenergetics of cancer cells (Wang et al., (2015) Nat. Chem. 7: 968-979). The ATP depletion activates 5 AMP-activated protein kinase (AMPK) which in turn increases the demand for fatty acid oxidation instead of lipogenesis (Wang et al., (2015) Nat. Chem. 7: 968-979). A similar disruption to ATP generation was observed when treating cancer cells in vitro with concentration over 100 μM of tetrathiomolybdate (TM), an FDA-approved copper chelator for Wilson's disease, where oxidative phosphorylation was decreased and cells exhibited increased dependency on glycolysis (Ishida et al., (2013) Proc. Natl. Acad. Sci. (U.S.A.) 110: 19507-19512; Kim et al., (2015) Sci. Rep. 5:14296).

### SUMMARY

Cancer cells often undertake aerobic glycolysis for bioenergetics, a phenomenon known as the Warburg effect. Triple negative breast cancer (TNBC), among many other types of cancer, also relies on fatty acid oxidation as an energy source. The present disclosure encompasses embodiments of a mitochondrial copper depleting strategy that exploits the potential vulnerability of this metabolic need of TNBC. The Copper-Depleting Nanoparticles (CDN) of the disclosure, a positively charged copper depleting complex, target mitochondria and deprive the cytochrome c oxidase of its copper co-factor. Inhibition of the electron transport chain complex IV compromises oxygen consumption and abrogates fatty acid oxidation, resulting in energy deficiency induced apoptosis of TNBC cells. The disruption of oxidative phosphorylation leads to a unique metabolic switch from fatty acid oxidation to glycolysis and upregulation of fatty acid synthesis. The CDN can report the copper depleting status through multimodal optical signal changes and effectively decrease the copper level in TNBC tumors to inhibit tumor growth with low toxicity and significantly prolonged survival.

One aspect of the disclosure encompasses embodiments of a nanoparticle comprising a self-reporting copper-depleting moiety (CDM) and a matrix, wherein the matrix comprises a semi-conducting polymer and a phospholipid-polyethylene glycol (PEG), wherein the CDM is embedded in or on the matrix.

In some embodiments of this aspect of the disclosure, the copper-depleting moiety (CDM) can be linked to a fluorescent moiety.

In some embodiments of this aspect of the disclosure, the copper-depleting moiety (CDM) can be N,N-bis(2-pyridinylmethyl)-1,2-ethanediamine.

In some embodiments of this aspect of the disclosure, the fluorescent moiety can be a tricarbocyanine.

In some embodiments of this aspect of the disclosure, the linkage of the CDM to the fluorescent moiety can be configured to allow the CDM, when bound to a copper ion, to quench a fluorescence emission from the fluorescent moiety.

In some embodiments of this aspect of the disclosure, the semi-conducting polymer can be a photoacoustic semi-conducting polymer.

Another aspect of the disclosure encompasses embodiments of a method of reducing the amount of free copper ions in the mitochondria of a target cell or population of target cells, the method comprising the step of delivering a nanoparticle to the interior of a target cell, wherein the nanoparticle comprises (a) a matrix comprising a semi-conducting polymer and a phospholipid-polyethylene glycol (PEG); and (b) a self-reporting copper chelator (CDM) embedded in or on the matrix, whereby copper ions in the mitochondria of the recipient cell chelate to the nanoparticle, thereby reducing the level of free copper in the mitochondria.

In some embodiments of this aspect of the disclosure, the copper-depleting moiety (CDM) can be linked to a fluorescent moiety, wherein the linkage of the CDM to the fluorescent moiety can be configured to allow the CDM, when bound to a copper ion, to quench a fluorescence emission from the fluorescent moiety.

In some embodiments of this aspect of the disclosure, the copper-depleting moiety (CDM) can be N,N-bis(2-pyridinylmethyl)-1,2-ethanediamine.

In some embodiments of this aspect of the disclosure, the fluorescent moiety can be a tricarbocyanine.

In some embodiments of this aspect of the disclosure, the semi-conducting polymer can be a photoacoustic semi-conducting polymer.

In some embodiments of this aspect of the disclosure, the target cell can be a cancer cell and the population of target cells can be in a tumor.

In some embodiments of this aspect of the disclosure, the target cell or the population of target cells can be in an animal or human subject tumor.

In some embodiments of this aspect of the disclosure, the method can further comprise the step of detecting a change in an optical signal generated from the nanoparticle by chelation of copper ions to the nanoparticle.

In some embodiments of this aspect of the disclosure, the method can further comprise the step of detecting a photoacoustic signal from the nanoparticle and determining the location of the nanoparticle within the animal or human subject.

Yet another aspect of the disclosure encompasses embodiments of a method of reducing at least one of the proliferation and the viability of a cancer cell by administering a self-reporting copper-depleting moiety (CDM) and a matrix, wherein the matrix comprises a semi-conducting polymer and a phospholipid-polyethylene glycol (PEG), wherein the CDM is embedded in or on the matrix, wherein copper ions in the mitochondria of the recipient cell are chelated by the CDM, thereby reducing the level of free copper in the mitochondria and reducing at least one of the proliferation and the viability of a cancer cell in the patient.

In some embodiments of this aspect of the disclosure, the method can further comprise copper-depleting moiety (CDM) is linked to a fluorescent moiety, wherein the linkage of the CDM to the fluorescent moiety is configured to allow the CDM, when bound to a copper ion, to quench a fluorescence emission from the fluorescent moiety, wherein the copper-depleting moiety (CDM) is N,N-bis(2-pyridinylmethyl)-1,2-ethanediamine, the fluorescent moiety is a tricarbocyanine and the semi-conducting polymer is a photoacoustic semi-conducting polymer.

In some embodiments of this aspect of the disclosure, the cancer can be Triple Negative Breast Cancer (TNBC).

In some embodiments of this aspect of the disclosure, the method can further comprise the step of detecting at least one of an optical signal from the nanoparticle generated after chelation of copper ions to the nanoparticle and detecting the presence of the cancer in the patient by detecting a change in the optical signal from the nanoparticle generated by chelation of copper ions to the nanoparticle and determining the location of the cancer in the patient.

In some embodiments of this aspect of the disclosure, the method can further comprise adjusting the level of a dose of a therapeutic agent administered to the patient in need thereof, whereby the change of the intensity of the optical signal indicates the level of the reduction of the proliferation or viability of the cancer cell caused by the therapeutic agent

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1G illustrate the design and characterization of CDN.

FIG. 1A illustrates the molecular components of CDN and the illustrated nanoparticle formulation. When binding with Cu(I) or Cu(II), the fluorescence of CDM is quenched but the photoacoustic signal from the semiconducting polymer remains unchanged, allowing for quantitative analysis of the chelation process.

FIG. 1B illustrates the UV-Vis spectrum of CDN. CDM absorption peaks at 610 nm and the polymer peaks at 1,100 nm.

FIG. 1C illustrates the Size and morphology of CDN measured by DLS (mean s.e.m., n=4 independent experiments) and TEM imaging (n=2 independent experiments, two images acquired per experiment). Inset, TEM image of CDN with uranyl acetate staining (scale bar, 100 nm). PDI, polydisperse index.

FIG. 1D illustrates the Zeta potential measurement of SPN with or without CDM loading (mean s.d., n=5 independent samples for SPN and n=4 independent samples for CDN).

FIG. 1E illustrates the fluorescence signal changes of CDN (CDM, 1 μM; Em, 740 nm) upon binding with Cu(I) or Cu(II) in buffered solution (mean±s.d., n=3 independent samples).

FIG. 1F illustrates the percentage of fluorescence signal of CDN solution (CDM, 5 μM) after mixing with various metal ions to that without metal addition (Na⁺, 5 mM; Mg²⁺, 5 mM; K⁺, 5 mM; Ca²⁺, 5 mM; Zn²⁺, 100 μM; Fe²⁺, 20 μM; Mn²⁺, 1 μM; Ni²⁺, 0.5 μM; Cd²⁺, 0.5 μM; Co²⁺, 0.5 μM) (mean±s.d., n=3 independent samples). Concentrations of the tested metal ions are selected based on the physiological abundancy. Measurement after CDN mixing with indicated physiologically abundant or trace metal ions and measurement after addition of extra 5 μM Cu(II) to previous mixture of CDM and metal ions are shown.

FIG. 1G illustrates a representative fluorescence imaging of CDN mixed with different equivalents of Cu(I) in the agar phantom (n=3 independent samples). Signal intensity is normalized based on the photoacoustic signal at 1,100 nm for each well. a.u., arbitrary units; d.nm, diameter in nanometers; eq, equivalent; F/Fblk, percentage of fluorescence signal after addition with metal ions relative to that before metal addition; p s⁻¹, photons per second; SP, semiconducting polymer; UV-Vis, UV-visible.

FIGS. 2A-2E illustrate that CDN induces TNBC cell death via intracellular copper depletion.

FIG. 2A illustrates representative optical images of MDA-MB-231luc cells after 24 h of incubation with CDN with or without different concentrations of copper supplements or excessive EDTA for copper deprivation (CDM, 1 μM). CDM fluorescence depicts copper binding to CDN and CCL-1 bioluminescence imaging depicts intracellular labile copper.

FIG. 2B illustrates MDA-MB-231 cell viability (percentage of cell control without treatment) after 24 h of incubation with different concentrations of CDN, CDM, TPA, TM or ATN224 in complete medium with 10% FBS measured by MTS assay (mean±s.e.m., n=3 biologically independent samples).

FIG. 2C illustrates MDA-MB-468 cell viability (percentage of cell control without treatment) after 24 h of incubation with different concentrations of CDN, CDM, TPA, TM or ATN224 in complete medium with 10% FBS measured by MTS assay (mean±s.e.m., n=3 biologically independent samples).

FIG. 2D illustrates the viability of MDA-MB-231 cells after 24 h of treatment with SPN or CDN with or without copper, zinc, iron or manganese addition (CDM, 1 μM; SPN, 50 μg ml⁻¹) measured by MTS assay (mean±s.d., n=3 biologically independent samples; P value from unpaired t-test, two-tailed).

FIG. 2E illustrates the viability of TNBC cells (MDA-MB-231 and MDA-MB-468) and normal human cells (MCF-10A, WI-38 and RWPE-1) after 24 h of treatment with CDN (CDM, 1 μM) in serum-free media (mean±s.e.m., n=3 biologically independent samples).

FIGS. 3A-3F illustrate that CDN inhibits mitochondria complex IV.

FIG. 3A illustrates the mitochondrial membrane potential of MDA-MB-231 cells after 24 h of incubation with CDN (CDM, 1 μM) with or without copper supplements (10 μM) or other control agents (chelator concentration, 1 μM; SPN concentration, 50 μg ml⁻¹), indicated by JC-1 staining (10 μg ml⁻¹). Red fluorescence that is emitted by aggregated JC-1 characterizes a high membrane potential while green fluorescence emitted by JC-1 monomer characterizes low membrane potential (scale bar, 50 μm, n=3 independent experiments).

FIG. 3B illustrates the COX activities of MDA-MB-231 and MDA-MB-468 cells after 24 h of incubation with CDN (CDM, 1 μM), TPA (1 μM) or ATN224 (5 μM) (mean±s.e.m., n=3 biologically independent samples; P value from unpaired t-test, two-tailed). Results are expressed as percentage of cell control without treatment.

FIG. 3C illustrates that, CDN inhibits cellular oxygen consumption in MDA-MB-231 cells. At 24 h after incubation with CDN (CDM, 1 μM) or control agents (TPA, 1 μM; ATN224, 1 μM or 5 μM; SPN, 50 μg ml⁻¹), the cellular OCR was measured by Seahorse analyzer. Oligomycin (1 μM) was introduced after 28 min, FCCP (1 μM) was introduced after 54 min and rotenone/antimycin A (0.5 μM) was introduced after 80 min (mean±s.e.m., n=8 biologically independent samples).

FIG. 3D illustrates that CDN inhibits cellular oxygen consumption in MDA-MB-468 (d) cells. At 24 h after incubation with CDN (CDM, 1 μM) or control agents (TPA, 1 μM; ATN224, 1 μM or 5 μM; SPN, 50 μg m^{l-1}), the cellular OCR was measured by Seahorse analyzer. Oligomycin (1 μM) was introduced after 28 min, FCCP (1 μM) was introduced after 54 min and rotenone/antimycin A (0.5 μM) was introduced after 80 min (mean±s.e.m., n=8 biologically independent samples).

FIG. 3E illustrates the inhibition of OCR mitigated with the addition of copper ion. MDA-MB-231 and MDA-MB-468 cells were incubated with 1 μM of CDN for 1 h with or without copper supplement (10 μM) (mean±s.e.m., n=6 biologically independent samples; P value from unpaired t-test, two-tailed).

FIG. 3F illustrates the cellular ATP level (as percentage of control with no treatment) after 24 h of incubation with CDN, TPA or ATN224 (chelator concentration, 1 μM) in FBS supplemented culture medium (mean±s.e.m., n=3 biologically independent samples; P value from unpaired t-test, two-tailed). NS, not significant.

FIGS. 4A-4H illustrate CDN-induced inhibition of mitochondrial oxidative phosphorylation (OXPHOS) changes metabolisms of TNBC cells.

FIGS. 4A and 4B illustrate that ECAR was elevated in both MDA-MB-231 (FIG. 4A) and MDA-MB-468 (FIG. 4B) cells at 24 h after incubation with CDN (CDM, 1 μM). After CDN treatment, MDA-MB-468 showed minimal cellular response to the subsequent injections of oligomycin (1 μM, introduced after 28 min), FCCP (1 μM, introduced after 54 min) and rotenone/antimycin A (0.5 μM, introduced after 80 min). MDA-MB-231 exhibited no response to oligomycin and rotenone/antimycin A. Unlike CDN, other control agent treatment (TPA, 1 μM; ATN224, 1 μM or 5 μM; SPN, 50 μg ml⁻¹) slightly decreased or did not impact the ECAR levels (mean±s.e.m., n=8 biologically independent samples).

FIG. 4C illustrates the glucose uptake (i) and lactate secreted into the medium (ii) over 24 h of incubation with ATN224 (5 μM) or CDN (CDM, 1 μM) (mean s.e.m., n=3 biologically independent samples; P value from unpaired t-test, two-tailed). Results are expressed as percentage of cell control without treatment.

FIG. 4D illustrates the glucose metabolism pathways affected by CDN treatment.

FIG. 4E illustrates the percentage of M+3 lactic acid over the total pool.

FIGS. 4F-4H illustrate the intensities of isotopologues of metabolites, including glutamine (FIG. 4F), glutamate (FIG. 4G) and UDP-N-acetylglucosamine (FIG. 4H), produced from ¹³C6-labeled glucose in vitro. Metabolites with isotopologues shown are written in red. Other important intermediate metabolites are written in black. Non-labeled ¹²Cs are shown as black-filled circles while labeled ¹³Cs are shown as red-filled circles. Raw intensities are normalized by protein concentration. Data are shown as mean±s.e.m. (n=5 biologically independent samples; P value from unpaired t-test, two-tailed). mpH, milli-pH units.

FIG. 5A-5F illustrate that CDN depletes copper efficiently in vivo.

FIG. 5A illustrates labile copper concentration detected by CCL-1 bioluminescence imaging after i.v. injection of CDN (CDM, 1.35 mg kg-1) in MDA-MB-231luc tumor-bearing mice (mean±s.e.m., n=3 independent animals).

FIG. 5B illustrates longitudinal monitoring of nanoparticle delivery and copper binding via optical imaging of CDN after i.v. injection of CDN. Photoacoustic signal in the tumor region (blue) reflects the accumulation and retention of CDN while fluorescence signal reports the copper binding to CDM (red) (mean s.d., n=3 independent animals).

FIGS. 5C and 5D illustrate the d-luciferin bioluminescence imaging (FIG. 5C) and CCL-1 imaging (FIG. 5D) of mice receiving SPN, TPA or CDN treatment (chelator dose, 1.35 mg kg⁻¹). MDA-MB-231luc tumor-bearing mice were intravenously administered with the indicated agents every 3 d with a total of five doses. Images were acquired at day 25 after the first injection (mean±s.e.m., n=5 independent animals; P value from unpaired t-test, two-tailed).

FIG. 5E illustrates labile copper levels in the tumor region for different treatment groups were quantified as CCL-1/d-luciferin flux ratio (mean±s.e.m., n=5 independent animals; P value from unpaired t-test, two-tailed).

FIG. 5F illustrates the in vivo mitochondrial membrane potential measured by MAL3 bioluminescence imaging. MDA-MB-231luc tumor-bearing mice were imaged before and after injection with PBS, SPN or CDN. The total photon flux of MAL3 bioluminescence signals was normalized by d-luciferin bioluminescence signal (mean±s.e.m., n=5 independent animals; P value from unpaired t-test, two-tailed). Effi, efficiency; PA, photoacoustic; p.i., post injection; sr, steradian.

FIGS. 6A-6G illustrate that CDN inhibits TNBC tumor growth.

FIG. 6A illustrates a treatment strategy for long-term survival study with MDA-MB-231 tumor-bearing mice. Six control and treatment groups were included in the study: control, SPN, TPA, ATN224, CDM and CDN. Mice received SPN, TPA, CDM or CDN (chelator dose, 1.35 mg kg⁻¹) via i.v. injection weekly and ATN224 via oral gavage daily (0.7 mg kg⁻¹ d⁻¹).

FIG. 6B illustrates a tumor growth curve of each individual mouse in different treatment groups (n=12 independent animals). In the CDM treatment group, * indicates individuals that received early euthanasia under the instruction of a veterinarian due to skin rashes.

FIG. 6C illustrates a survival curve of different treatment groups up to 70 d after the initial treatment.

FIG. 6D illustrates a tumor growth curve of MDA-MB-468 tumor-bearing mice during CDN and control agent treatment (mean s.e.m., n=5 independent animals). At day 40 after initial treatment, tumor burden in the CDN treatment group was significantly smaller than that in the nontreated control group (P=0.016), SPN group (P=0.003), TPA group (P=0.004) and ATN224 group (P=0.004) (P value from unpaired t-test, two-tailed). At 3 d after the seventh dose, mice were killed and tumors were subjected to metabolite analysis.

FIG. 6E illustrates glucose levels were determined (mean s.d., n=5 independent animals; P value from unpaired t-test, two-tailed).

FIG. 6F illustrates lactate levels were determined (mean s.d., n=5 independent animals; P value from unpaired t-test, two-tailed).

FIG. 6G illustrates alanine levels were determined (mean s.d., n=5 independent animals; P value from unpaired t-test, two-tailed).

FIGS. 7A-7E illustrate the design and characterization of fCDN.

FIG. 7A illustrates the molecular components of fCDN and the illustrated nanoparticle formulation. Fluorescent polymer fSP serves as FRET energy donor and CDM serves as an energy acceptor. When binding with Cu(I) or Cu(II), the fluorescence of CDM is quenched and the FRET is abolished, resulting in an increase of the polymer signal and decrease of the CDM signal. This ratiometric change depicts the copper binding to fCDN.

FIG. 7B illustrates the UV-Vis spectrum of fCDN, showing polymer peaks at 453 nm and CDM absorption peaks at 610 nm.

FIG. 7C illustrates the DLS size measurement of fCDN (mean±s.d., n=4 independent samples).

FIG. 7D illustrates the emission spectra of fCDN upon binding with different equivalents of Cu(II) in solution (Ex: 453 nm).

FIG. 7E illustrates the fluorescence ratiometric response of CDN to different equivalents of Cu(II) (Data points were extracted from one set of spectrum measurement).

FIGS. 8A-8C illustrate serum stability of CDN.

FIG. 8A illustrates the ratio of fluorescence emission intensity at 540 nm and 740 nm of fCDN (Ex: 453 nm, CDM concentration of 1.5 μM) after 24 h incubation with serum with or without 500 μM EDTA for the removal of labile copper or HEPES buffer (10 mM, pH 7.4) at 37° C. (mean±s.e.m., n=3 independent samples).

FIG. 8B illustrates the CDM fluorescence intensity when excited at 610 nm after 24 h incubation with serum with or without 500 μM EDTA at 37° C. (mean±s.e.m., n=3 independent samples). Results are presented as ratio percentage to the intensity before incubation (F₀).

FIG. 8C illustrates the Ceruloplasmin (Cp) activity in the serum with or without incubation with CDN for 24 h at 37° C. (mean s.e.m., n=3 independent samples). Results are presented as ratio percentage to the Cp activity (mU/mL) before incubation.

FIG. 9A illustrates the quantification of fluorescence images of MDA-MB-231^{luc} cells after 24 h incubation with CDM with or without different concentrations of copper supplements or excessive EDTA for copper deprivation (CDM: 1 μM). Results were shown as percentage of signal intensity of those in CDN treatment group (mean s.d., n=3 independent experiments).

FIG. 9B illustrates the quantification of luminescence images of MDA-MB-231^{luc} cells after 24 h incubation with CDM with or without different concentrations of copper supplements or excessive EDTA for copper deprivation (CDM: 1 μM). Results were shown as percentage of signal intensity of those in CDN treatment group (mean s.d., n=3 independent experiments).

FIG. 9C illustrates representative CCL-1 bioluminescence imaging of 4T1^{luc} cells after 24 h incubation with SPN, CDN, ATN224 or TPA (chelator concentration: 1 μM, SPN concentration: 50 μg/ml).

FIG. 9D illustrates the photon flux (n=2 independent experiments).

FIG. 10 is a series of representative confocal microscopy images of MDA-MB-231 and MDA-MB-468 cells after 24 h incubation with fCDN with or without 10 μM of Cu(II) supplement or 500 μM of EDTA (CDM: 1 μM). The green channel showed fluorescence from fSP and the red channel showed the FRET fluorescence from CDM when exciting at 488 nm. The copper content is indicated as the green/red ratio and presented in processed pseudo-colored images. (scale bar: 50 μm) (n=3 independent experiments).

FIG. 11A shows a western blot analysis of cleaved caspase 3 found after 24 h incubation with CDN (CDM: 1 μM) by, but not with ATN224 treatment (5 μM) in MDA-MB-468 and MDA-MB-231 cells. Lane 1, 4: control group without treatment; 2, 5: ATN224 group (5 μM); 3, 6: CDN group (CDM: 1 μM). Two experiments with were repeated independently with similar results.

FIG. 11B shows a western blot analysis of apoptosis induced by CDN treatment rescued with BID inhibitor, BI-6C9. Unlike CDN, IACS 010759 and BAY 872243 did not induce apoptosis of MDA-MB-231 cells at 24 h after incubation at 1 μM concentration. Lane 1, 6: control group without treatment; 2, 7: CDN group (CDM: 1 μM); 3, 8: CDN with BI-6C9 (CDM: 1 μM, BI-6C9: 100 μM); 4, 9: IACS 010759 (1 μM); 5, 10: BAY 87-2243 (1 μM). Two experiments were repeated independently with similar results.

FIG. 12 shows 4T1 cell viability (percentage of cell control without treatment) after 24 h incubation with different concentrations of CDN, TPA or ATN224 in complete medium with 10% FBS measured by MTS assay (mean±s.e.m., n=3 biologically independent samples).

FIG. 13A shows WI-38 cell viability (percentage of cell control without treatment) after 24 h incubation with different concentrations of CDN or CDM in serum-free medium (supplemented with EGF) measured by MTS assay (mean±s.e.m., n=3 biologically independent samples).

FIG. 13B shows RWPE-1 cell viability (percentage of cell control without treatment) after 24 h incubation with different concentrations of CDN or CDM in serum-free medium (supplemented with EGF) measured by MTS assay (mean±s.e.m., n=3 biologically independent samples).

FIG. 13C shows MCF-10A cell viability (percentage of cell control without treatment) after 24 h incubation with different concentrations of CDN or CDM in serum-free medium (supplemented with EGF) measured by MTS assay (mean±s.e.m., n=3 biologically independent samples).

FIGS. 14A and 14B illustrate inhibition on the cell invasion upon treatment with various control agents and CDN, determined by Matrigel in vitro invasion assay for (FIG. 14A) MDA-MB-231 and (FIG. 14B) MDA-MB-468 (CDM: 1 μM, SPN: 50 μg/ml). Invaded cell number was normalized by the cell viability measured by MTS study. Data is presented as mean±s.e.m. (P values from unpaired t test, two-tailed). For MDA-MB-231 cells, n=3 biologically independent samples for blank, SPN, CDN with copper, TPA, and ATN224 group; n=5 biologically independent samples for CDN group. For MDA-MB-468 cells, n=4 biologically independent samples for blank and SPN group; n=5 biologically independent samples for CDN group.

FIGS. 15A-15C illustrate the representative subcellular localization of CDN determined by co-staining of organelle and CDN.

FIG. 15A illustrates representative confocal microscopy images of cells co-incubated with Mitotracker (Green) and CDN (CDM: 1 μM, red) for 30 min.

FIG. 15B illustrates representative confocal microscopy images of cells incubated with CDN (CDM: 0.1 μM, 4 h). Cells were then washed with HBSS for 3 times and stained with ER-tracker Green for 30 min.

FIG. 15C illustrates representative confocal microscopy images of cells incubated with CDN (CDM: 0.1 μM, red) for 1 h. Cells were pre-incubated with CellLight® Early Endosomes-GFP (Green) (scale bar: 20 μm). Three experiments were repeated independently with similar results.

FIG. 16A illustrates the quantification of JC-1 staining in FIG. 9A. JC-1 red signal to green signal ratio of CDN treatment group is significantly lower than control (P<0.0001), SPN (P<0.0001), with copper supplement (P=0.008), TPA (P<0.0001) and ATN224 group (P<0.0001). The ROI analysis was performed out of 7 different views of cells (approximately 50 cells per view) from 3 independent experiments. Results are presented as mean±s.e.m.; P value was from unpaired t test, two-tailed.

FIG. 16B illustrates a Mitotracker-staining experiment showing that mitochondrial membrane integrity was compromised after CDN treatment. MDA-MB-231 was incubated with different concentrations of CDN for 24 h then stained with Mitotracker Green (Green: MT-G) and DAPI (blue) (Scale bar: 50 μm). Experiments were repeated twice independently with similar results.

FIG. 17 illustrates mRNA sequencing results showing a major downregulation of gene sets for the subunits of cytochrome c oxidase (except for COX6B2 at 15 h) and its copper chaperone and co-chaperone proteins after treatment with CDN (1 μM).

FIGS. 18A-18D illustrate that the addition of MitoTEMPO up to 5 μM will not affect the cell viability of MDA-MB-231 and MDA-MB-468 cells in medium (FIG. 18A) with or (FIG. 18B) without FBS supplement. When co-incubation with CDN (CDM: 1 μM) for 24 h, addition of MitoTEMPO did not rescue (FIG. 18C) MDA-MB-231 or (FIG. 18D) MDA-MB-468 cells in medium with or without FBS supplement. (mean±s.e.m., n=3 biologically independent samples) FIGS. 19A and 19B illustrate the intensities of isotopologues of metabolites, (FIG. 19A) 4-oxoproline, (FIG. 19B) N-acetyl-glutamate, produced from ¹³C₆-labeled glucose in vitro. Raw intensities are normalized by protein concentration. Data are shown as mean±s.e.m. (n=5 biologically independent samples, P values from unpaired t test, two-tailed).

FIG. 20 illustrates the labile copper concentration detected by CCL-1 bioluminescence imaging after i.v. injection of CDN (1.35 mg/kg) in MDA-MB-231^{luc} tumor-bearing mice. At each time point, mice were intraperitoneally injected with CCL-1 (6 mg/kg) and imaged with IVIS Spectrum.

FIG. 21 illustrates a representative 3D reconstruction of the tumor region (grey: B mode ultrasound, red: photoacoustic at 1100 nm) at different time points after i.v. injection of CDN (1.35 mg/kg) in MDA-MB-231 tumor-bearing mice.

FIG. 22 illustrates digital D-luciferin and CCL-1 bioluminescence imaging of mice in different treatment groups at day 25 after the first injection. MDA-MB-231^{luc} tumor-bearing mice were divided into four groups: control, SPN control, TPA and CDN (n=5 mice for each group). TPA and CDN group receive i.v. injection (chelator dose: 1.35 mg/kg) every three days with a total of five doses. Mice in SPN group received same SPN dose as the CDN group.

FIGS. 23A-23C illustrate the effect of copper depletion on mitochondrial membrane potential (ΔΨm) of MDA-MB-231^{luc} tumor xenografts measured with bioluminescent ΔΨm-specific MAL3 probe.

FIGS. 23A and 23B illustrate the design of the mitochondrial-activated luciferin (MAL) probe. The MAL probe consists of two components, a triphenylphosphine-caged luciferin probe (TPP-CL, red-colored ball) and an azido-triphenylphosphine reagent (azido-TPP, blue-colored ball), both of which are targeted to mitochondria because of the triphenylphosphine (TPP) group, similar to other compounds widely used for selective delivering of small molecules to mitochondria. The “click” reaction (Staudinger ligation) between the reagents results in the uncaging of a luciferin derivative (green-colored ball) that in the presence of luciferase results in production of photon flux that can be imaged and quantified by a CCD camera or standard plate reader. This technology is easily applicable for noninvasive imaging and monitoring of small changes in ΔΨm both in vitro and in vivo in a longitudinal fashion.

FIG. 23A illustrates the chemical reaction of MAL components.

FIG. 23B illustrates the theoretical illustration of the cellular distribution of MAL reagents in different cellular compartments (extracellular—1×; cytosolic—3×-10×; mitochondrial matrix—100×-500×).

FIG. 23C illustrates the timeline of the experiment. On day 0 mice received i.v. injection of either CDN, SPN, or PBS (n=5 independent mice). MAL3 probe consists of TPP-CL2 and AzidoTPP1 reagents that are injected sequentially with a 20 h interval. The size of the tumors was assessed by injection of concentrated solution of luciferin right after imaging with MAL3.

FIG. 24 illustrates the representative confocal microscopy images of tumor and liver slices from control and fCDN administered (CDM dose: 1.35 mg/kg) MDA-MB-231 tumor bearing mice (scale bar: 100 μm). Slices from 6 independent animals were imaged and showed similar results.

FIG. 25A illustrates tumor growth curve of 4T1 tumor bearing mice in different treatment groups. Data are shown as mean±s.e.m. (n=5 independent animals). For the treatment plan, 4T1 tumor bearing mice were divided into three groups: control, TPA and CDN (n=5 mice for each group). TPA and CDN groups received i.v. injection (chelator dose: 1.35 mg/kg) weekly days with a total of five doses.

FIG. 25B illustrates a photograph of tumors harvested at day 32 after initial treatment (four tumors in the blank control group were harvested at day 22 due to the large sizes). For the treatment plan, 4T1 tumor bearing mice were divided into three groups: control, TPA and CDN (n=5 mice for each group). TPA and CDN groups received i.v. injection (chelator dose: 1.35 mg/kg) weekly days with a total of five doses.

FIG. 26 illustrates mRNA sequencing results showing a major upregulation of gene sets involved in fatty acid synthesis and cholesterol synthesis after 24 h treatment with CDN. All measurements were taken from distinct samples.

FIG. 27 illustrates a schematic synthesis of CDM.

FIG. 28 illustrates a schematic synthesis of photoacoustic semiconducting polymer (SP).

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

Prior to describing the various embodiments, the following definitions are provided and should be used unless otherwise indicated.

### Definitions

The terms “administering” and “administration” as used herein refer to a process by which a therapeutically effective amount of a compound of the disclosure or compositions contemplated herein are delivered to a subject for prevention and/or treatment purposes.

Compositions are administered in accordance with good medical practices considering the subject's clinical condition, the site and method of administration, dosage, patient age, sex, body weight, and other factors known to physicians.

The term “cancer,” as used herein shall be given its ordinary meaning and is a general term for diseases in which abnormal cells divide without control. Cancer cells can invade nearby tissues and can spread through the bloodstream and lymphatic system to other parts of the body. There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue, such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it with differing processes that have gone awry. Solid tumors may be benign (not cancerous) or malignant (cancerous). Different types of solid tumors are named for the type of cells that form them. Examples of solid tumors are sarcomas, carcinomas, and lymphomas. Leukemias (cancers of the blood) generally do not form solid tumors. Representative cancers include, but are not limited to, bladder cancer, breast cancer, colorectal cancer, endometrial cancer, head and neck cancer, leukemia, lung cancer, lymphoma, melanoma, non-small-cell lung cancer, ovarian cancer, prostate cancer, testicular cancer, uterine cancer, and cervical cancer.

The term “cell or population of cells” as used herein refers to an isolated cell or plurality of cells excised from a tissue or grown in vitro by tissue culture techniques. Most particularly, a population of cells refers to cells in vivo in a tissue of an animal or human.

The term “contacting a cell or population of cells” as used herein refers to delivering a probe according to the present disclosure to an isolated or cultured cell or population of cells, or administering the probe in a suitable pharmaceutically acceptable carrier to the target tissue of an animal or human. Administration may be, but is not limited to, intravenous delivery, intraperitoneal delivery, intramuscularly, subcutaneously, or by any other method known in the art. One advantageous method is to deliver directly into a blood vessel leading into a target organ or tissue such as a prostate, and so reducing dilution of the probe in the general circulatory system.

The term “delivering to a cell” as used herein refers to the direct targeting of a cell with a small molecule compound by systemic targeted delivery for in vivo administration, or by incubation of the cell or cells with said effector ex vivo or in vitro.

The term “dye” as used herein refers to any reporter group whose presence can be detected by its light absorbing or light emitting properties. For example, Cy5 is a reactive water-soluble fluorescent dye of the cyanine dye family. Cy5 is fluorescent in the red region (about 650 to about 670 nm). It may be synthesized with reactive groups on either one or both of the nitrogen side chains so that they can be chemically linked to either nucleic acids or protein molecules. Labeling is done for visualization and quantification purposes. Cy5 is excited maximally at about 649 nm and emits maximally at about 670 nm, in the far-red part of the spectrum; quantum yield is 0.28. FW=792. Suitable fluorophores(chromes) for the probes of the disclosure may be selected from, but not intended to be limited to, fluorescein isothiocyanate (FITC, green), cyanine dyes Cy2, Cy3, Cy3.5, Cy5, Cy5.5, Cy7, Cy7.5 (ranging from green to near-infrared), Texas Red, and the like. Derivatives of these dyes for use in the embodiments of the disclosure may be, but are not limited to, Cy dyes (Amersham Bioscience), Alexa Fluors (Molecular Probes Inc.), HILYTE® Fluors (AnaSpec), and DYLITE® Fluors (Pierce, Inc).

The term “fluorescence” as used herein refers to a luminescence that is mostly found as an optical phenomenon in cold bodies, in which the molecular absorption of a photon triggers the emission of a photon with a longer (less energetic) wavelength. The energy difference between the absorbed and emitted photons ends up as molecular rotations, vibrations or heat. Sometimes the absorbed photon is in the ultraviolet range, and the emitted light is in the visible range, but this depends on the absorbance curve and Stokes shift of the particular fluorophore.

The terms “fluorescence quencher” or “quencher” as used herein refers to molecules that interfere with or absorb the fluorescence emitted by a nearby fluorophore.

The term “fluorophore” as used herein refers to any reporter group whose presence can be detected by its light emitting properties.

The term “modulate” refers to the activity of a composition to affect (e.g., to promote or retard) an aspect of cellular function, including, but not limited to, cell growth, proliferation, apoptosis, and the like.

The term “modulated detectable signal” as used herein refers to a detectable signal emitted by a label moiety that is reduced in intensity or otherwise changed such as, but not limited to, a change in wavelength such that the modulated signal is detectably distinct from a unmodulated signal. A modulated signal can be, for example, a quenched signal where some or all of the energy of the unmodulated signal is absorbed by a second label moiety so that the modulated signal is less intense than the original signal. Alternatively, for example, a first signal from a first label moiety may be a stimulant for a second label moiety to emit a signal of a different wavelength.

The term “nanoparticle” As used herein refers to a particle having a diameter of between about 1 and about 1000 nm, preferably between about 100 nm and 1000 nm, and most preferably between about 50 nm and 700 nm. Similarly, by the term “nanoparticles” is meant a plurality of particles having an average diameter of between about 50 and about 1000 nm.

It will be understood by one of ordinary skill in the art that when referring to a population of nanoparticles as being of a particular “size”, what is meant is that the population is made up of a distribution of sizes around the stated “size”. Unless otherwise stated, the “size” used to describe a particular population of nanoparticles will be the mode of the size distribution (i.e., the peak size). By reference to the “size” of a nanoparticle is meant the length of the largest straight dimension of the nanoparticle. For example, the size of a perfectly spherical nanoparticle is its diameter.

The terms “quench” or “quenches” or “quenching” or “quenched” as used herein refer to reducing the signal produced by a molecule. It includes, but is not limited to, reducing the signal produced to zero or to below a detectable limit. Hence, a given molecule can be “quenched” by another molecule and still produce a detectable signal albeit the signal is greatly reduced.

The terms “fluorescence quencher” or “quencher” as used herein refers to molecules that interfere with or absorb the fluorescence emitted by a nearby fluorophore.

The term “subject” generally refers to an individual who will receive or who has received treatment (e.g., administration of a compound of the disclosure, and optionally one or more other agents) for a condition characterized by a cancer. In certain aspects, a subject may be a healthy subject. Typical subjects for treatment include persons afflicted with or suspected of having or being pre-disposed to a disease disclosed herein, or persons susceptible to, suffering from or that have suffered a disease disclosed herein.

The term “fluorescent acceptor molecule” as used herein refers to any molecule that can accept energy emitted as a result of the activity of a bioluminescent donor protein, and re-emit it as light energy.

The terms “fluorescence quencher” or “quencher” as used herein refer to a molecule that interferes with the fluorescence emitted by a fluorophore or bioluminescent polypeptide.

This quencher can be selected from non-fluorescent aromatic molecules, to avoid parasitic emissions. Exemplary quenchers include, but are not limited to, Dabsyl or a BLACK HOLE QUENCHER® that are non-fluorescent aromatic molecules that prevent the emission of fluorescence when they are physically near a fluorophore. The quencher can also be, but is not limited to, a fluorescent molecule, for example TAMRA (carboxytetramethylrhodamine). A particularly advantageous quencher suitable for use in the compositions of the disclosure is a modified dye such as IR-775-COOH. When the quencher is a fluorescent dye, its fluorescence wavelength is typically substantially different from that of the reporter dye.

The terms “quench” or “quenches” or “quenching” or “quenched” as used herein refer to reducing the signal produced by a molecule. It includes, but is not limited to, reducing the signal produced to zero or to below a detectable limit. Hence, a given molecule can be “quenched” by, for example, another molecule and still produce a detectable signal, albeit the size of the signal produced by the quenched molecule can be smaller when the molecule is quenched than when the molecule is not quenched.

The term “detectable signal emitter” as used herein refers, for the purposes of the specification or claims, to a label molecule that is incorporated indirectly or directly into a nanoparticle, wherein the label molecule facilitates the detection of the nanoparticle in which it is incorporated, for example when the nanoparticle of the disclosure is at a site of inflammation and activated by interaction between the nanoparticle or the quencher component thereof and a RONS. Thus, “detectable signal emitter” is used synonymously with “label molecule”.

The term “NH₂-functionalized conjugated polymer” as used herein refers to a nanoparticle formed by co-condensing one or more types of monomer to form a polymer and wherein on the outer surface of said nanoparticle are located amine groups that are available for conjugating with another molecular entity that may have such as a reactive carboxyl group thereon.

The term “detectable” refers to the ability to detect a signal over the background signal.

The term “detecting” refers to detecting a signal generated by one or more photoacoustic probes. It should be noted that reference to detecting a signal from a photoacoustic probe also includes detecting a signal from a plurality of photoacoustic probes. In some embodiments, a signal may only be detected that is produced by a plurality of photoacoustic probes. Additional details regarding detecting signals (e.g., acoustic signals) are described below.

The term “acoustic detectable signal” is a signal derived from a probe of the present disclosure that absorbs light and converts absorbed energy into thermal energy that causes generation of acoustic signal through a process of thermal expansion. The acoustic detectable signal is detectable and distinguishable from other background acoustic signals that are generated from the host. In other words, there is a measurable and statistically significant difference (e.g., a statistically significant difference is enough of a difference to distinguish among the acoustic detectable signal and the background, such as about 0.1%, 1%, 3%, 5%, 10%, 15%, 20%, 25%, 30%, or 40% or more difference between the acoustic detectable signal and the background) between acoustic detectable signal and the background. Standards and/or calibration curves can be used to determine the relative intensity of the acoustic detectable signal and/or the background.

The detectable signal is defined as an amount sufficient to yield an acceptable image using equipment that is available for pre-clinical use. A detectable signal maybe generated by one or more administrations of the probes of the present disclosure. The amount administered can vary according to factors such as the degree of susceptibility of the individual, the age, sex, and weight of the individual, idiosyncratic responses of the individual, the dosimetry, and the like. The amount administered can also vary according to instrument and digital processing related factors.

The term “optical energy” as used herein refers to electromagnetic radiation between the wavelengths of about 350 nm to about 800 nm and which can be absorbed by the dyes or cellulose-based nanoparticles of the embodiments of the photoacoustic probes of the disclosure. The term “optical energy” may be construed to include laser light energy or non-laser energy.

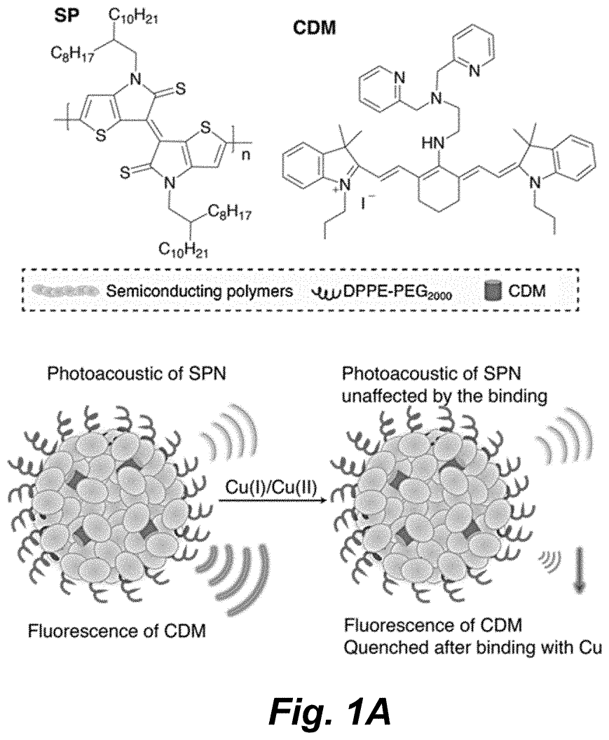

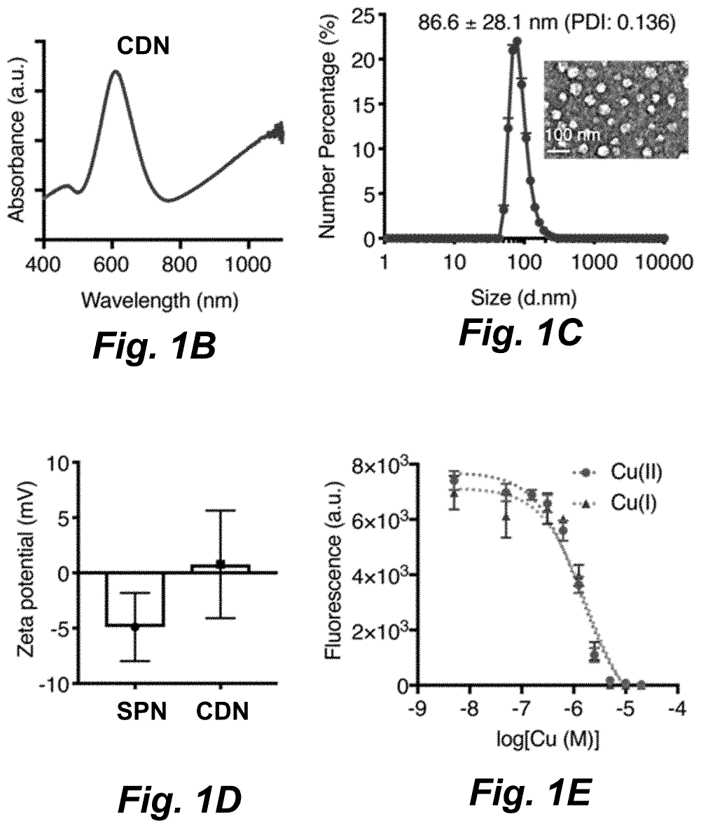

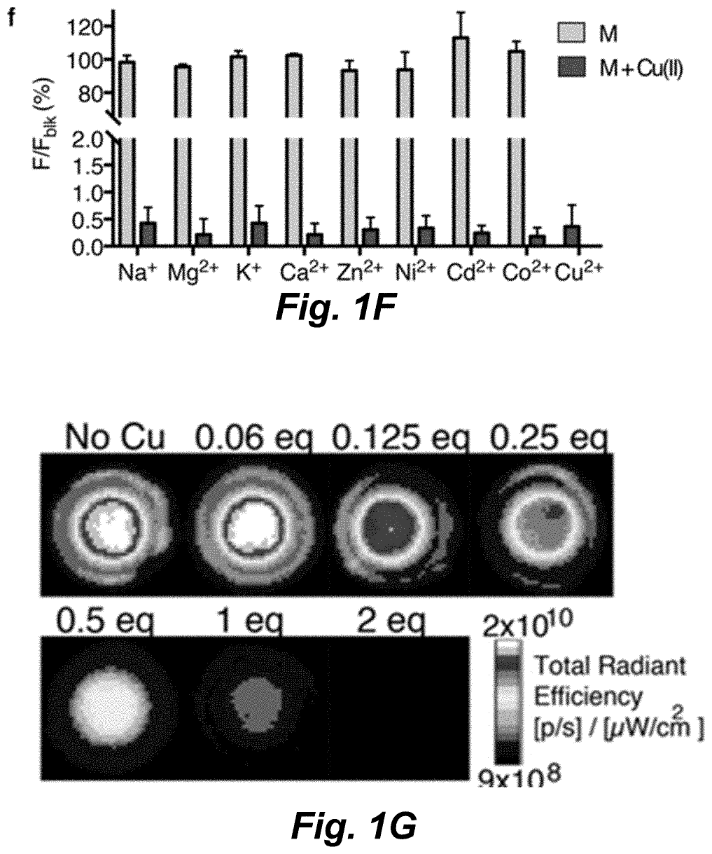

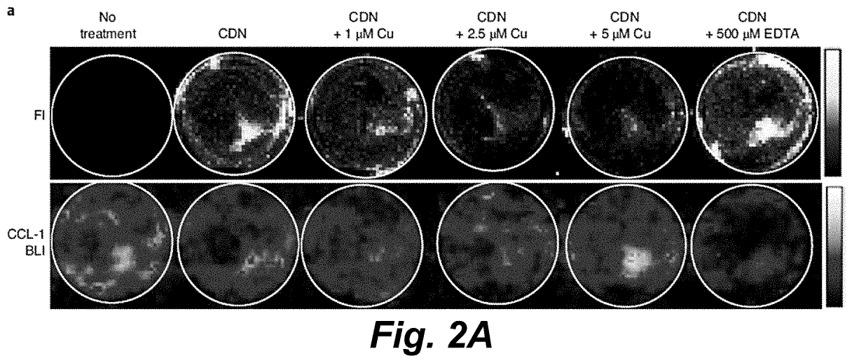

In particular, it is understood that the nanoparticle core of the compositions of the disclosure can comprise a semiconducting polymer that may emit an acoustic signal when irradiated by a suitable incident energy. The semiconducting polymers suitable for use in the compositions of the disclosure may advantageously include, but are not limited to, poly(cyclopentadithiophene-alt-benzothiadiazole) (PCPDTBT) (Muhlbacher et al., (2006) Adv. Mater. 18: 2884-2889) and poly(acenaphthothienopyrazine-alt-benzodithiophene) (PATPBDT) as shown in FIG. 1A. Other suitable semiconducting polymers include, but are not limited to, such as poly[4,6-(dodecyl-thieno[3,4-b]thiophene-2-carboxylate)-alt-2,6-(4,8-dioctoxylbenzo[1,2-b:4,5-b]dit, poly[N-90-heptadecanyl-2,7carbazoe-alt-3,6-bis(thiophen-5-yl)-2,5-dioctyl-2,5-dihydropyrrolo[3,4]pyrrole-1,4-dione], poly{4,8-bis[(2-ethylhexyl)oxy]benzo[1,2-b:4,5-b′]dithiophene-2,6-diyl-alt-3-fluoro-2-[(2-ethylhexyl], poly[2,6(4,4′bis(ethylhexyl)dithieno[3,2-b:2′,3′-d]silole)-alt-(1,3-(5-octyl-4H-thieno[3,4-c]pyrrole], or their derivatives.

The term “acoustic signal” as used herein refers to a sound wave produced by one of several processes, methods, interactions, or the like (including light absorption) that provides a signal that can then be detected and quantitated with regard to its frequency and/or amplitude. The acoustic signal can be generated from one or more photoacoustic probes. In some embodiments, the acoustic signal may need to be the sum of each of the individual photoacoustic probes or groups of photoacoustic probes. In some embodiments, the acoustic signal can be generated from a summation, an integration, or other mathematical process, formula, or algorithm, where the acoustic signal is from one or more photoacoustic probes. The summation, the integration, or other mathematical process, formula, or algorithm can be used to generate the acoustic signal so that the acoustic signal can be distinguished from background noise and the like.

The term “photoacoustic imaging” as used herein refers to a biomedical imaging modality based on the photoacoustic effect. The photoacoustic effect refers to when light energy is transformed into kinetic energy of the sample. This results in local heating, and thus a pressure wave or sound. In photoacoustic imaging, non-ionizing laser pulses are delivered into biological tissues. Some of the delivered energy will be absorbed and converted into heat, leading to transient thermoelastic expansion and thus ultrasonic emission at MHz frequencies. The generated ultrasonic waves are detected by ultrasonic transducers to form images. Since optical absorption has been associated with physiological properties, such as hemoglobin concentration or oxygen saturation, the ultrasonic emission (i.e. photoacoustic signal) reveals physiologically specific optical absorption contrast. 2D or 3D images of the targeted areas are possible.

### Discussion

The present disclosure uses a mitochondrial copper depleting strategy to exploit the metabolic vulnerability of TNBC to shut down the OXPHOS and eventually induce apoptosis as a result of energy deprivation. Embodiments of a copper-depleting moiety embedded semiconducting polymer-based nanoplatform (CDN or CDN) have been developed to deplete copper in tumor with minimal side effects to healthy tissues. Mechanistically, the CDN's positive surface charge allows for favorable mitochondrial accumulation and local depletion of copper. Consequently, COX is inactivated. As a result, the mitochondrial OXPHOS activity is shut down and TNBC cells adopt a metabolic switch from fatty acid oxidation to glycolysis with largely decreased ATP generation. The inhibited fatty acid oxidation also leads to the upregulation of fatty acid synthesis in the TNBC cells that further strain the energy supply. Healthy cells are less susceptible due to their lower demand for copper and less uptake of the vesicle. In addition to the metabolic reprogramming capacity, the nanoparticles (CDNs) of the disclosure possess self-reporting of copper chelation and selective delivery to tumor, making it a highly potent therapeutic agent for treating TNBC.

Metabolic reprogramming is a central hallmark of cancer. The “Warburg effect” (Koppenol et al., (2011) Nat. Rev. Cancer 11: 325-337) describes how cancer cells often undergo glycolysis under aerobic condition for energy production (Weinberg & Chandel (2015) Nat. Chem. Biol. 11: 9-15; Vyas et al., (2016) Cell 166: 555-566) and precursor generation for biosynthetic pathways essential for cell survival and proliferation, such as serine synthesis and fatty acid synthesis and pentose phosphate pathway for NADPH and nucleotide production (Gouw et al., (2019) Cell Metab. 30: 556-572; Vyas et al., (2016) Cell 166: 555-566). However, not all cancers have preference on glycolysis for bioenergetics and there is an increasing emphasis on tumor and cancer cell subpopulation's reliance on mitochondrial OXPHOS. Cancer types including breast cancer (Park et al., (2016) Cell Rep. 14: 2154-2165; Hoy et al., (2017) Trends Mol. Med. 23: 381-392), prostate cancer (Liu: Y. (2006) Prostate Cancer Prostatic Dis. 9: 230-234), metastatic ovarian cancer (Nieman et al., (2011) Nat. Med. 17: 1498-1503; Schild et al., (2018) Cancer Cell 33: 347-354), and glioblastoma (Duman et al., (2019) Cell Metab. 30: 274-289) have been found to import lipid from adjacent adipocytes and employ fatty acid oxidation as their primary energy source. Patient tumor immunohistochemistry staining has shown that TNBC has the highest percentage of negative staining of pyruvate kinase M2 isoform (PKM2) (Israelsen et al., (2013) Cell 155: 397-409), the enzyme catalyzing the final step in glycolysis and generating ATP, suggesting glycolysis is not considered indispensable for TNBC.

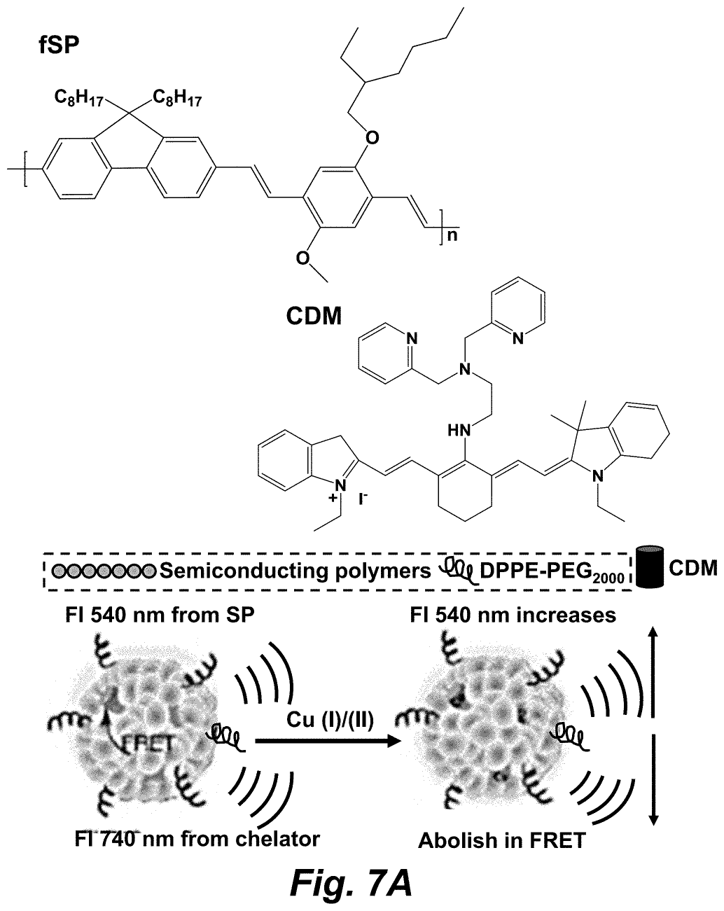

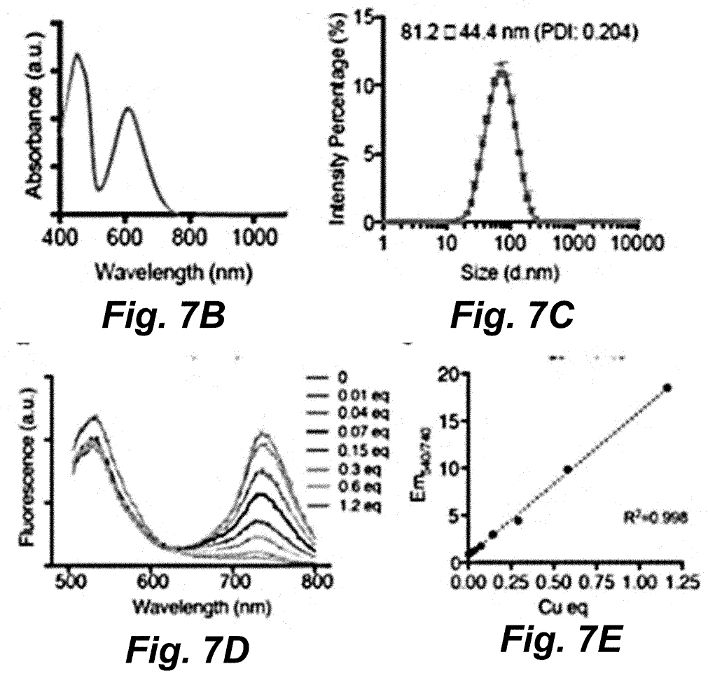

Design, formulation and characterization of CDNs of the disclosure: A CDN of the disclosure was formulated by incorporating a potent self-reporting copper chelator (CDM) into a PEGylated nanoparticle matrix that consists of semiconducting polymers and phospholipid-polyethylene glycol (PEG) (semiconducting polymer nanoparticle, SPN) (FIGS. 1A and 7A). The CDM, N,N-bis(2-pyridinylmethyl)-1,2-ethanediamine linked with a fluorescent dye. The fluorescent dye can be, but is not limited to, a tricarbocyanine. This linkage is configured to allow a near-infrared (NIR) fluorescence signal from the tricarbocyanine to be quenched when copper binds to the CDM, thereby capable of a providing real-time feedback of the chelation.

Two embodiments of the SPNs of the disclosure were employed to generate CDNs with different optical properties. As shown in FIG. 1B, SPN is photoacoustic at 1100 nm, and can be non-invasively tracked in vivo by photoacoustic imaging. When coupled with fluorescence signal changes from CDM, the optical signals of the system can report the amount of CDN as well as the copper binding status in the organ of interest. fCDN (FIGS. 7A-7E) is a fluorescence resonance energy transfer (FRET)-based system, whereby the semiconducting polymer acts as the FRET donor and the CDM linked thereto is the acceptor (FIG. 7A). fCDN detects the copper binding through fluorescence ratiometric imaging (FIGS. 7D and 7E), which is suitable for cellular mechanism study and ex vivo quantification.

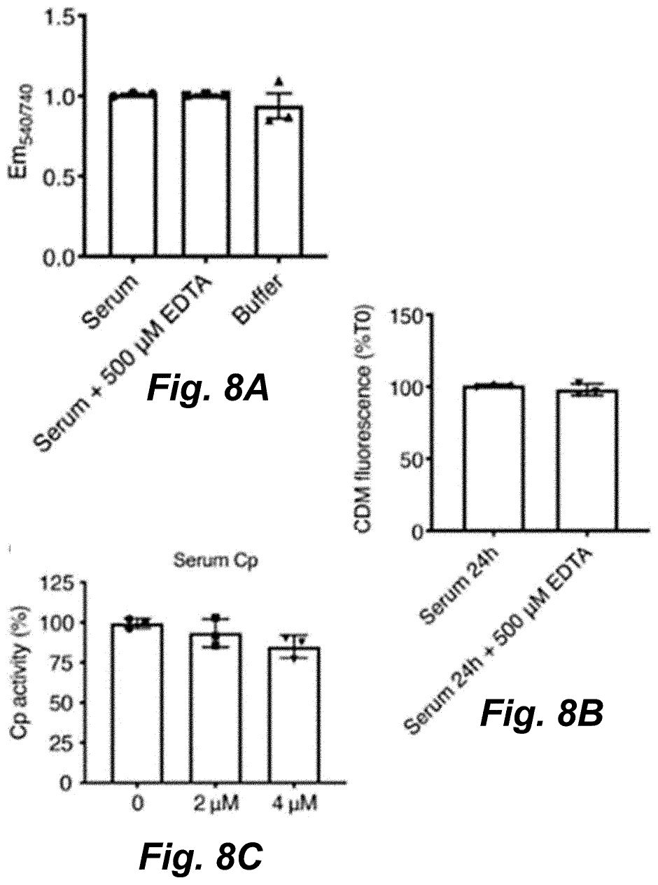

The CDNs of the disclosure were prepared with a nano-emulsion method as reported previously (Pu et al., (2014) Nat. Nanotechnol. 9: 233-239). Both formulations had high loading capacity of CDM with a w:w ratio of up to 0.28:1 (CDM:SP). The resulting CDNs were monodispersed spherical nanoparticles with hydrodynamic size around 85 nm (FIGS. 1C and 7C) as determined by dynamic light scattering (DLS) and transmission electron microscopy (TEM). The loading of CDM in the matrix yielded a slightly positive surface change on the nanocomplex (ζ potential of 0.77±4.86 mV for CDN, 0.91±4.83 mV for fCDN versus −4.89±3.08 mV for SPN, FIG. 1D) that afforded the mitochondria targeting ability of CDN. Once formulated, the CDN was stable in storage for at least 45 days. Minimal CDM leakage (1.38%) was observed in serum after 24 h incubation at 37° C. (FIG. 8).

CDN binds with Cu(I) and Cu(II) promptly in buffered solution, resulting in the decrease of CDM fluorescence at 740 nm (FIG. 1E). The dissociation constant (K_{d}) of CDN to Cu(I) was 1.80±0.12 μM and 1.36±0.07 μM to Cu(II). CDN is highly specific for copper ions and physiologically abundant metal ions were not observed to compete with copper binding when tested at the normal body content level (FIG. 1F). CDN reports the level of copper via changes in the optical signals, as demonstrated by the agar phantom imaging with IVIS Spectrum for CDM fluorescence and VisualSonics Vevo for photoacoustics for signal normalization (FIG. 1G).

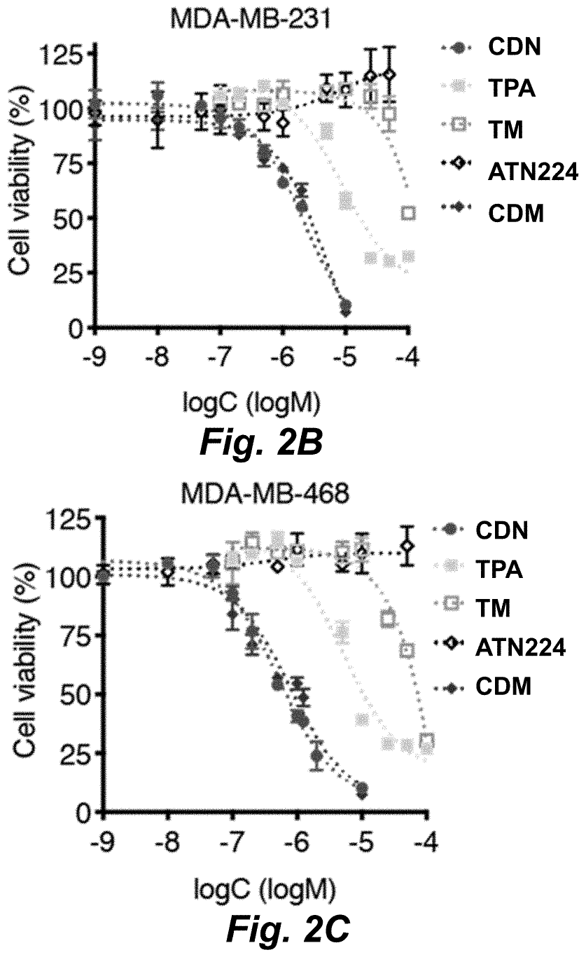

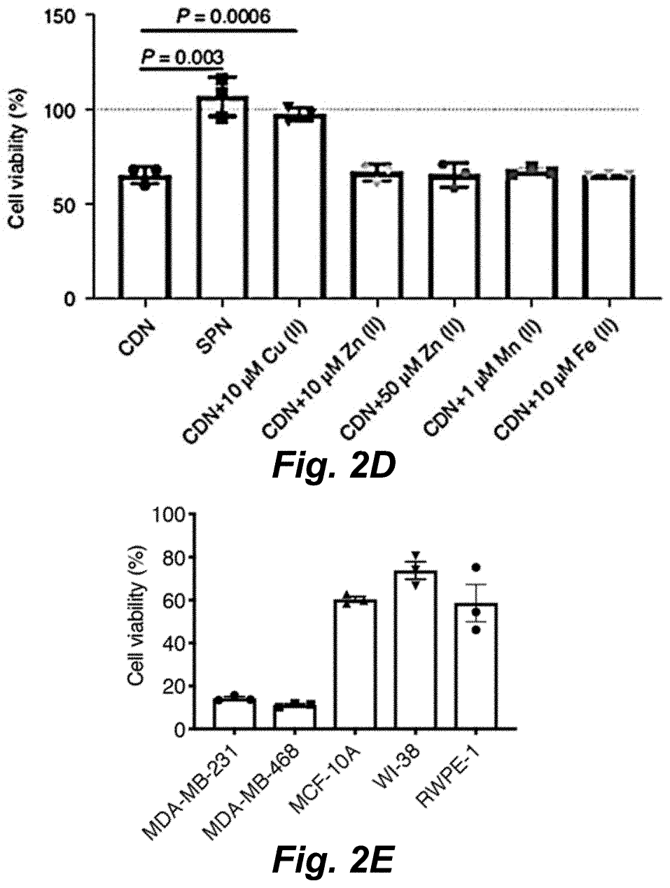

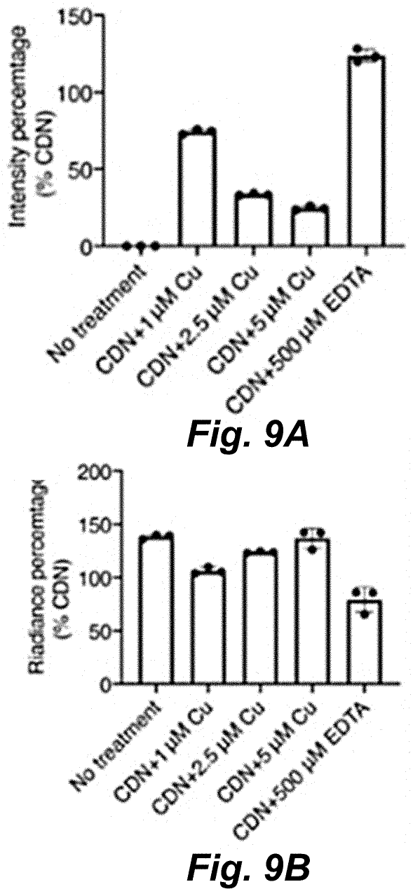

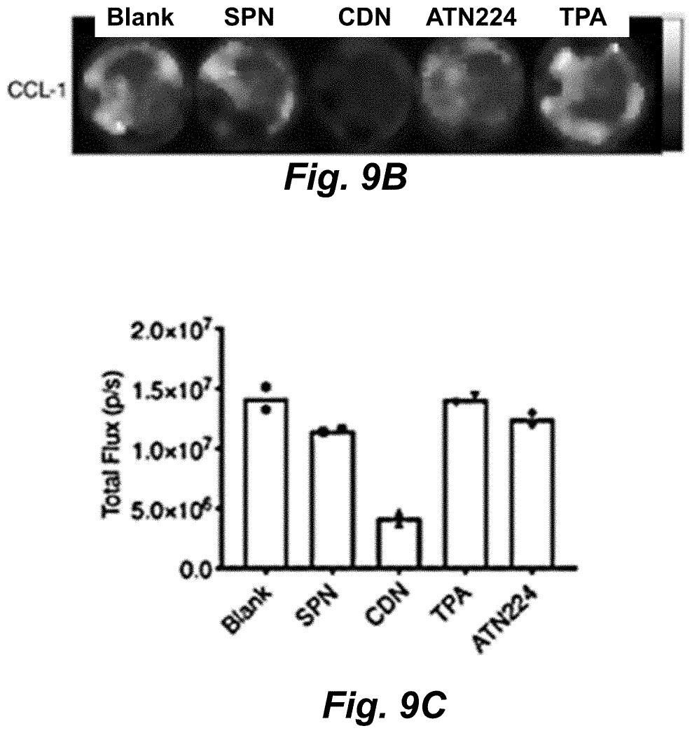

CDN depletes intracellular copper and induces cell death: Whether CDN could be taken up by TNBC cells and deplete copper intracellularly was tested. To independently measure the labile copper concentration in cells, a bioluminescent probe CCL-1 reported previously (Heffern et al., (2016) Proc. Natl. Acad. Sci. USA 113: 14219-14224) was used which is a D-luciferin analog caged by tris[(2-pyridyl)-methyl]amine (TPA) ligand. Upon oxidative cleavage with Cu(I), CCL-1 generates D-luciferin that subsequently reacts with firefly luciferase to produce bioluminescence. To test the cellular uptake and copper binding, MDA-MB-231^{luc} or 4T1^{luc} cells (both of which were stably transfected with firefly luciferase) was co-incubated with CDN and different concentrations of copper (FIGS. 2A and 9A-9D). Compared to the control group, addition of CDN decreased the labile copper concentration as indicated by a lower bioluminescent signal from CCL-1. The CDM fluorescence from CDN was partially quenched (CDM: 1 μM). Extra copper supply rescued the depleting effect, as shown by the observed increase in CCL-1 luminescence and decrease in CDM fluorescence.

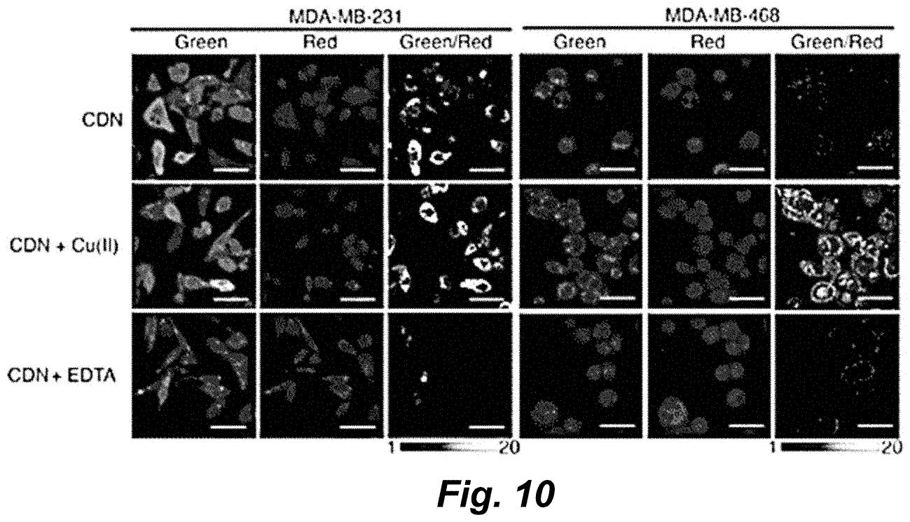

Meanwhile, the copper-starved group with excess ethylenediaminetetraacetic acid (EDTA) showed the highest CDM fluorescence (123.7±4.1% of the CDN group) and lowest CCL-1 luminescence (78.6±11.7% of the CDN group). With fCDN, intracellular copper binding was monitored in individual cells using confocal microscopy. Both MDA-MB-231 and MDA-MB-468 cells showed increased green/red ratios after 24 h incubation with fCDN and Cu(I) supplement in comparison to the control without Cu(I) supplement (FIG. 10). On the contrary, the EDTA supplement dramatically decreased the green/red ratio. This result again validates that the CDN of the disclosure can report its own copper binding status.

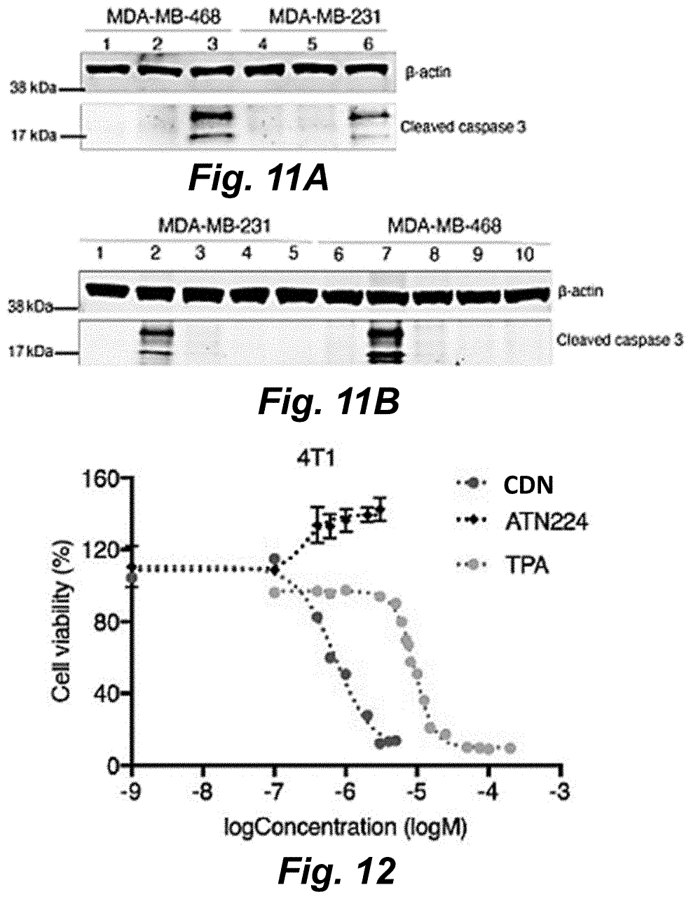

Depletion of intracellular copper by CDN induced apoptosis in MDA-MB-231 and MDA-MB-468 cells, evidenced by the presence of cleaved caspase 3 after incubation (FIGS. 11A and 11B). CDN-induced apoptosis can be effectively inhibited by BID inhibitor BI-6C9, indicating that the apoptosis was associated with the release of cytochrome c or Smac from mitochondria (Supplementary FIG. 11B). It appears that CDN was more efficient in stopping copper trafficking due to the mitochondria targeting in cancer cells and encapsulating effect of copper ions in the nanostructure.

Three small molecular copper chelators were used to test this hypothesis: free CDM; TPA, a copper chelator that structurally resembles the chelating group in CDM without its mitochondria-targeting ability; and TM (or its choline salt ATN224). The cell viability after control agents or CDN treatment was tested with [3-(4,5-dimethylthiazol-2-yl)-5-(3-carboxymethoxyphenyl)-2-(4-sulfophenyl)-2H-tetrazolium (MTS) assay (FIGS. 2B and 2C) and the trypan blue exclusion method. When treating TNBC cells in the presence of serum for 24 h, as shown in FIG. 2B, in MDA-MB-231, the half-maximum inhibitory con-centration (IC₅₀) of CDN is 2.31±0.45 μM, in comparison with 5.48±1.33 μM for CDM, 9.25±2.19 μM for TPA and over 100 μM for TM. For MDA-MB-468 cells, the IC₅₀ is 0.53±0.05 μM for CDN, 0.78±0.16 μM for CDM, 5.34±1.12 μM for TPA and around 46.3 μM for TM (FIG. 2C). Lower IC₅₀ for CDN was also observed in 4T1 cells (FIG. 12).

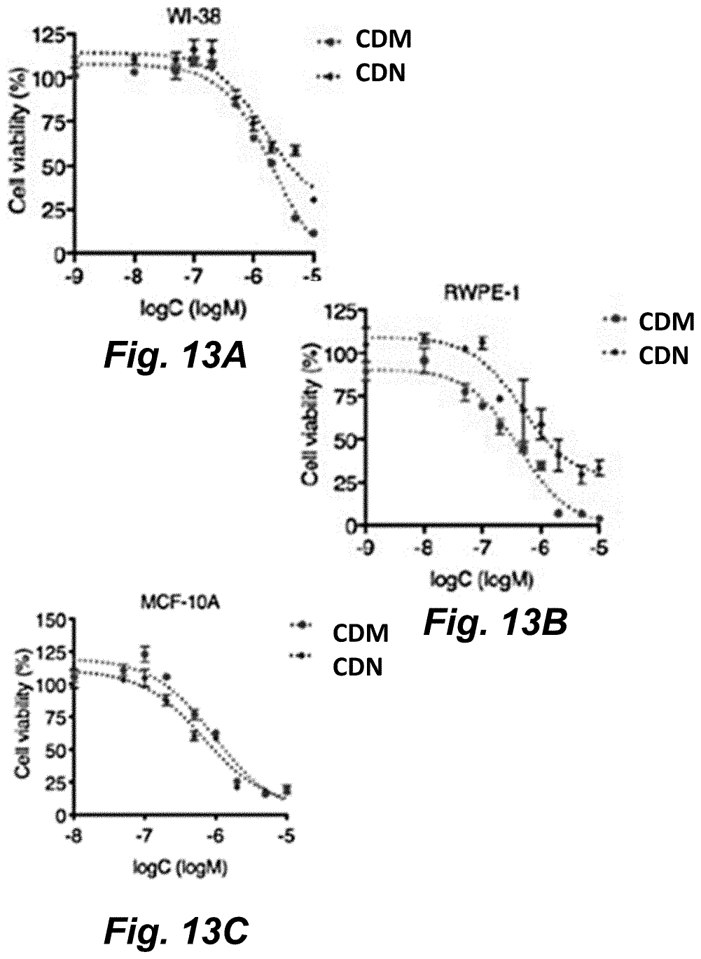

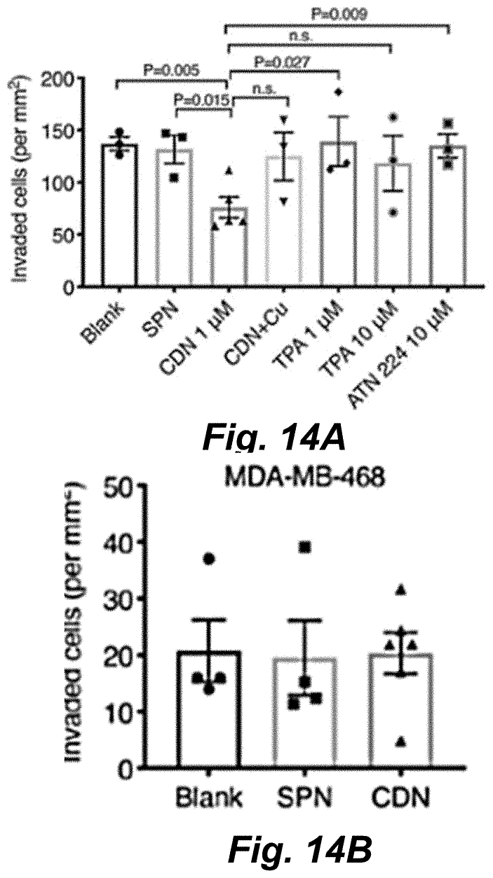

The cytotoxic effect due to the depletion of copper but not zinc, iron or manganese was further validated in a ‘metal remedy’ experiment. As shown in FIG. 2D, 10 μM copper supplement countered the cytotoxic effect of CDN; however, the addition of iron, manganese or zinc did not remediate the cytotoxicity effect. The copper ion titration study showed that the restoration of the cell viability was dependent on the concentration of copper co-incubated with CDN. Most importantly, CDN is less toxic to normal healthy cells than to TNBC cells. After incubation with CDN (1 μM) in the absence of serum for 24 h, the viability was 60.30±2.24% for healthy mammary gland epithelial cell MCF-10A, 73.77±6.98% for lung fibroblast cell WI-38 and 58.59±15.00% for prostate epithelial cell RWPE-1, all significantly higher than for MDA-MB-231 cell (14.29±1.25%) and MDA-MB-468 cell (11.24±0.88%). CDM became less toxic to WI-38 and RWPE-1 cells when loaded into SPN, likely due to the lower uptake of nanoparticle in both cells. Little difference was observed in MCF-10A (FIGS. 13A-13C). Other than the cytotoxic effect, copper depletion also decreased the cell motility of highly invasive MDA-MB-231 cells (FIGS. 14A and 14B).

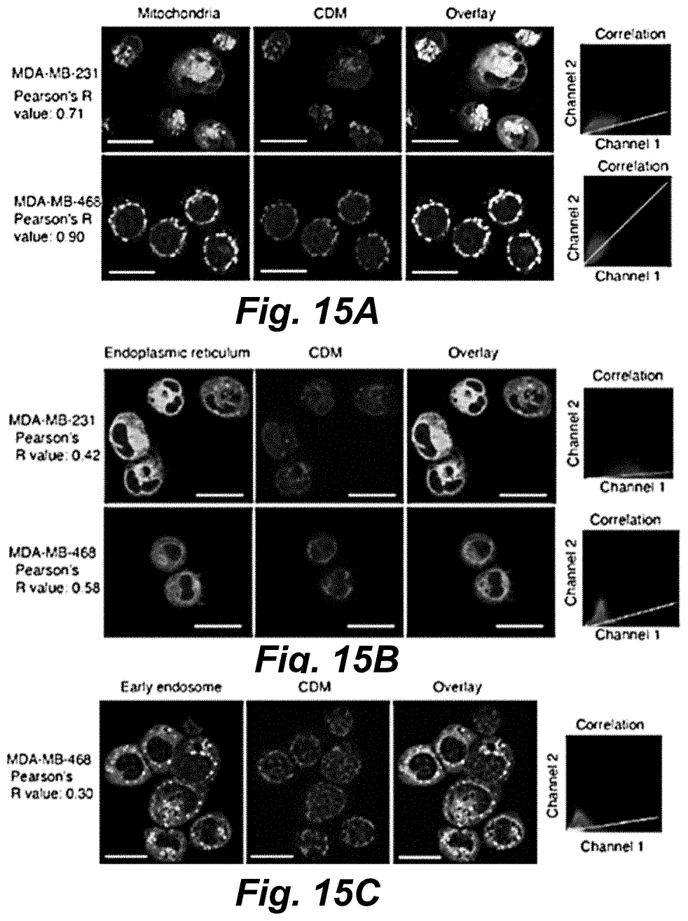

CDN inhibits OXPHOS in TNBC cells with a direct impact on COX activity: The subcellular localization of CDN with various organelle markers was determined. In accordance with one hypothesis, CDN accumulated in mitochondria but not early endosomes or endoplasmic reticulum (FIG. 15A-15C).

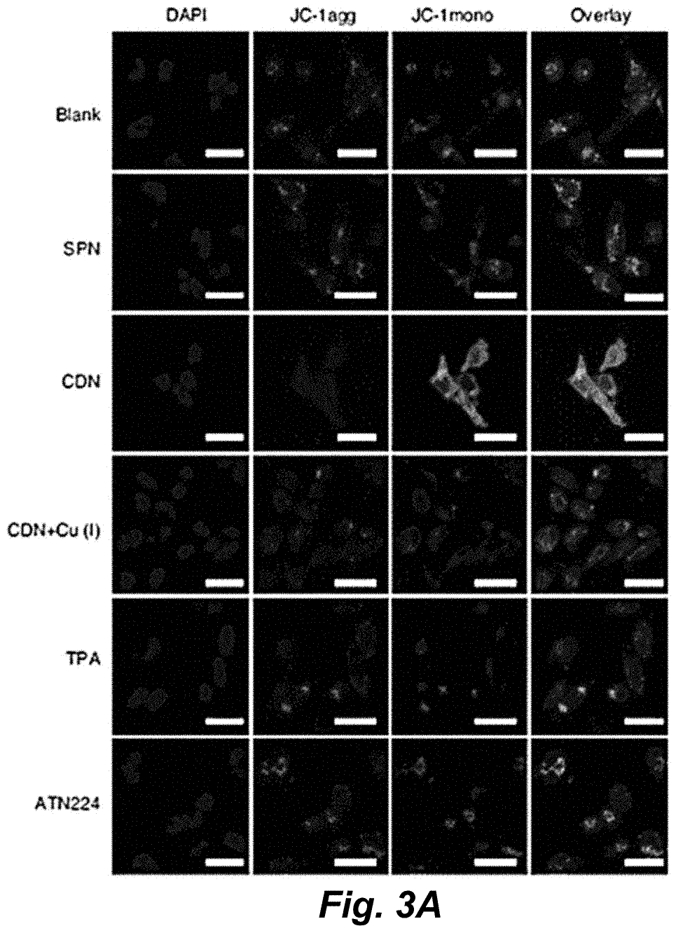

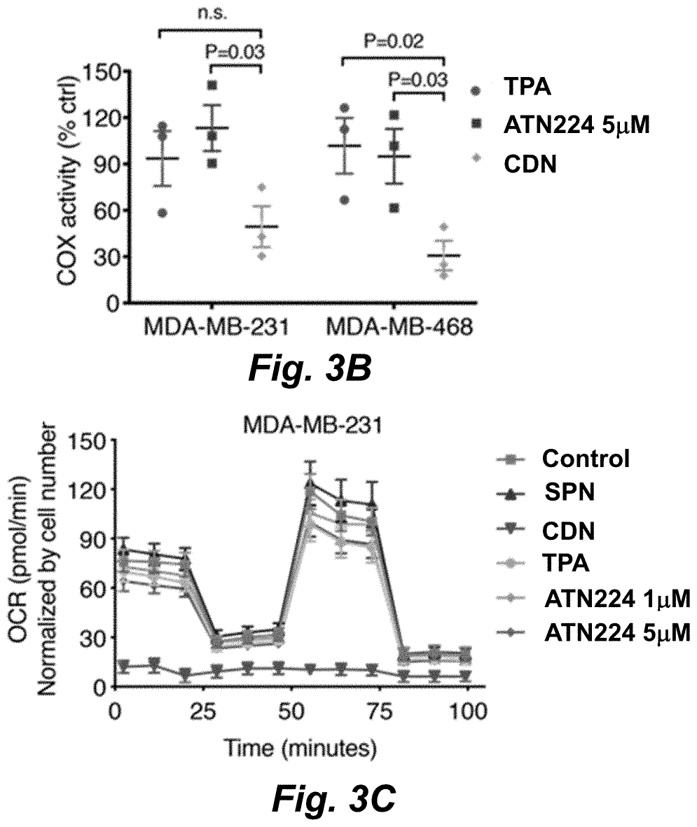

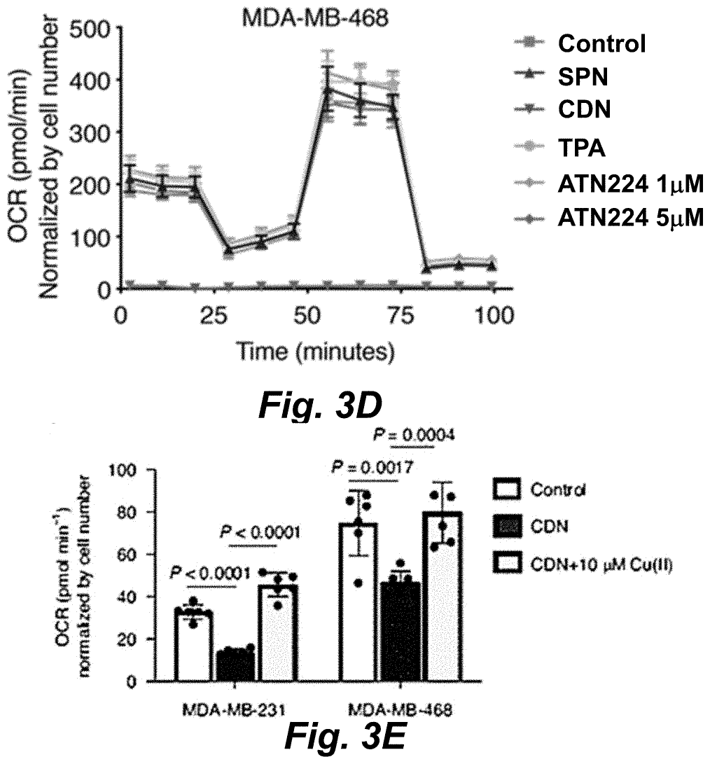

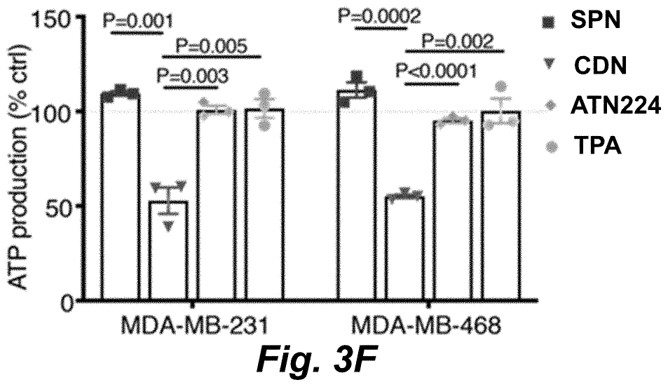

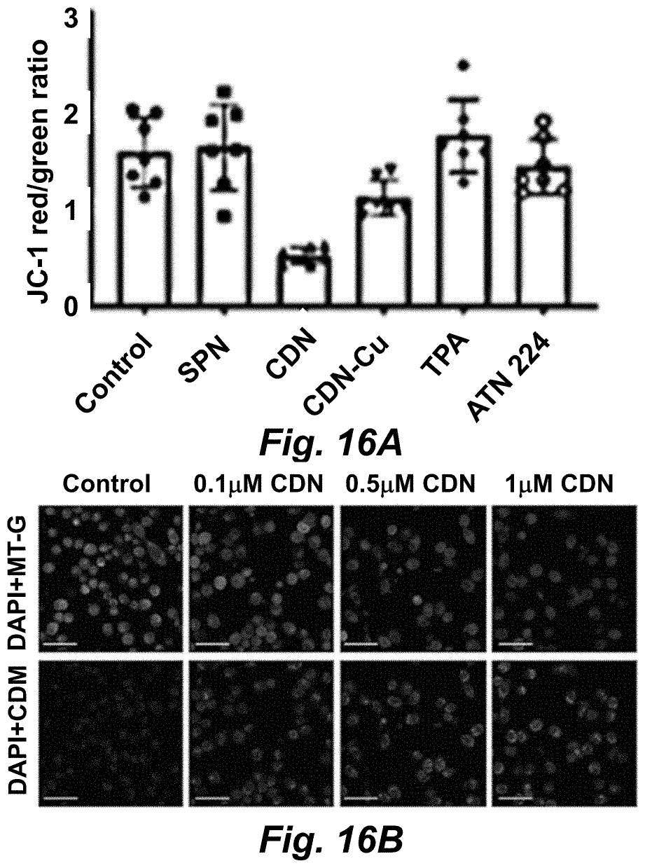

Next was investigated if CDN disrupted the mitochondrial function and metabolic pathways. JC-1 dye was used to evaluate the mitochondrial integrity. JC-1 accumulates and aggregates on intact mitochondrial membrane, yielding a red colored J-aggregate emission; when the membrane is compromised with a low potential, the dye spreads and exists as green fluorescent monomers. As shown in FIGS. 3A and 16A, MDA-MB-231 cells had strong red fluorescence in the mitochondria when treated with control agents, indicating that the mitochondria remained undamaged. On the contrary, cells treated with CDN produced low red fluorescence and strong green fluorescence, indicating that the mitochondrial membrane potential was compromised. The addition of copper prevented the mitochondrial damage and restored the red fluorescence. As a direct result of copper depletion in the mitochondria, COX activity after CDN treatment decreased to 49.44±13.28% as compared to control without any treatment for MDA-MB-231 cells and 30.70±9.55% as compared to control for MDA-MB-468 cells (FIG. 3B). This inhibition of COX activity by mitochondrial copper depletion is more potent than general limiting of the copper pool using treatment with TPA or ATN224.

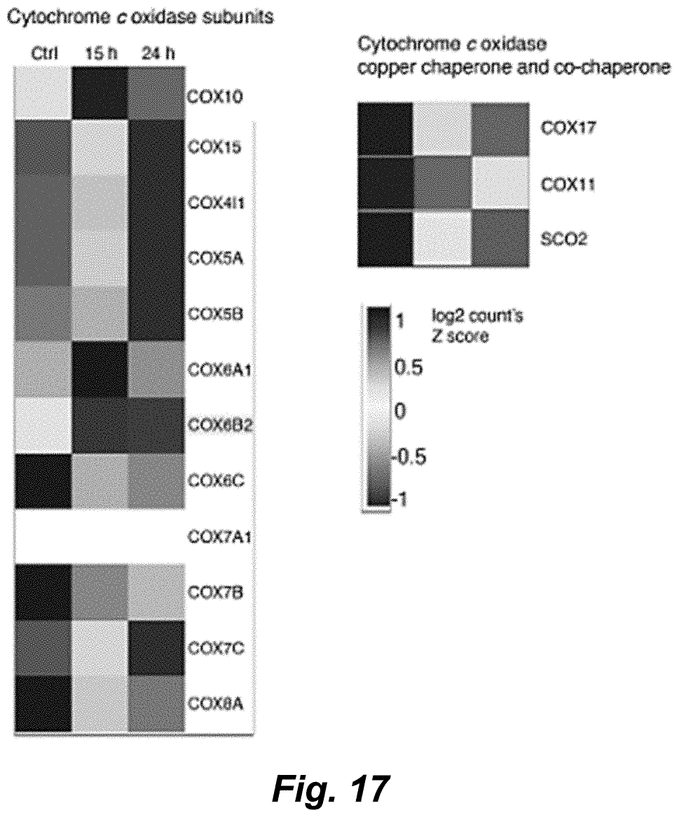

CDN treatment resulted in transcriptional downregulation of COX subunits and chaperone and co-chaperone proteins that deliver copper to mitochondria. As shown in FIG. 17, other than COX6B2, mRNA levels of most COX units went down. COX17, COX11 and SCO2 are important for copper delivery and assembly and maturation of COX. Their mRNA levels decreased after 15 h of CDN treatment.

Inhibition on the COX activity by CDN resulted in subsequent shutdown of cellular OXPHOS: Measurement of the oxygen consumption rate (OCR) showed that treating with CDN for 24 h resulted in a complete inhibition of OCR that is as potent as well-characterized OXPHOS inhibitors (e.g. rotenone/antimycin A). In comparison, other control agents at the same or higher concentration merely impacted on the OCR in both MDA-MB-231 and MDA-MB-468 cells (FIGS. 3C and 3D). The OCR inhibition by CDN was restored to the normal baseline level when extra copper ion was provided (FIG. 3E). An acute OCR inhibition response was also observed in both cell lines when injecting either CDN or free CDM.
OCR inhibition was examined at different concentrations of CDN: at a concentration higher than 1 μM, OCR was largely inhibited by CDN, and cells were not responsive to the subsequent injection of oligomycin (ATP synthase inhibitor) and carbonyl cyanide 4-(trifluoromethoxy)phenylhydrazone (FCCP, uncoupling agent that collapses the proton gradient); at relatively lower concentrations, OCR response showed a mixed effect of respiratory chain component inhibition and proton leak due to mitochondria uncoupling, suggesting that CDN also induced mitochondrial membrane disruption. Moreover, cellular ATP production significantly decreased in both TNBC cell lines treated with CDN (FIG. 3F; 52.8±6.9% of control for MDA-MB-231, 55.1±1.0% of control for MDA-MB-468). ATP levels in the cells treated with all control agents remained unaffected.

To further elucidate the cytotoxic mechanism of CDN, established OXPHOS inhibitors BAY 87-2243 and IACS-010759 were used as a comparison for in vitro experiments (Ellinghaus et al., (2013) Cancer Med. 2: 611-624 (2013); Molina et al., (2018) Nat. Med. 24: 1036-1046 (2018)). CDN caused higher cytotoxicity in both MDA-MB-231 and MDA-MB-468 cells at 24 h after incubation compared with the two complex I inhibitors. Cleaved caspase 3 levels after CDN treatment were also significantly higher than those after treatment of BAY 87-2243 and IACS-010759; the two inhibitors barely induced apoptosis at 24 h post-incubation (FIG. 12B). However, although CDN significantly reduced the OCR in both cell lines as compared with nontreated control, the blocking effect was not as potent as the two inhibitors, BAY 87-2243 and IACS-010759, which almost completely inhibited the oxygen consumption after 1 h of incubation.

All three agents were able to significantly elevate the extracellular acidification rate (ECAR) levels. Of the three agents, IACS-010759 had the least impact on the mitochondrial membrane integrity, and BAY 87-2243 treatment resulted in partially compromised membrane integrity. This difference may contribute to the slightly higher cytotoxicity of BAY 87-2243 compared with IACS-010759. CDN treatment has proven to disrupt mitochondrial membrane in JC-1 staining and MitoTracker staining experiments (FIGS. 3A and 17). Altogether, these results indicated that CDN-enabled copper depletion interfered with OXPHOS-related cellular bioenergetics by directly inhibiting the COX activity and damaging the mitochondrial membrane potential.

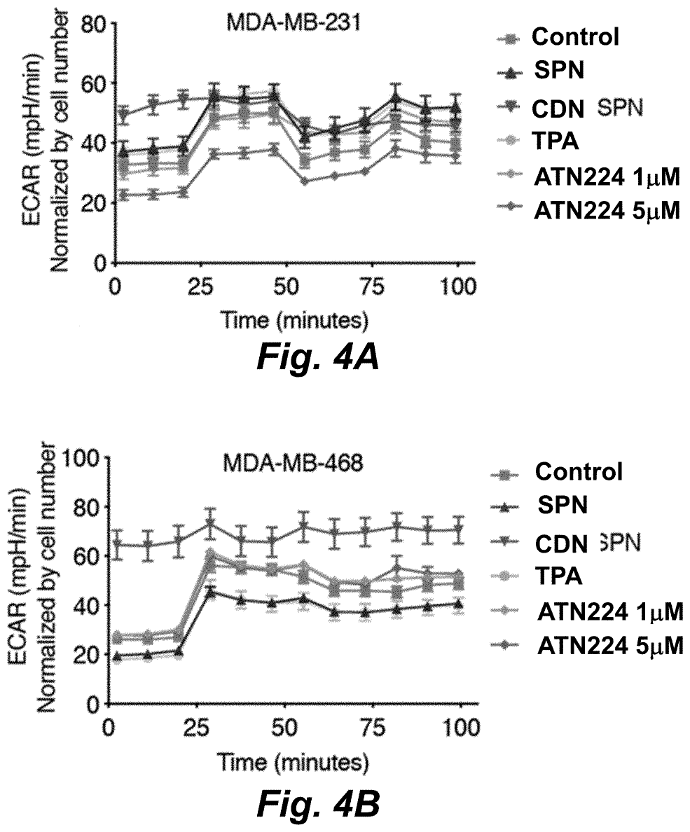

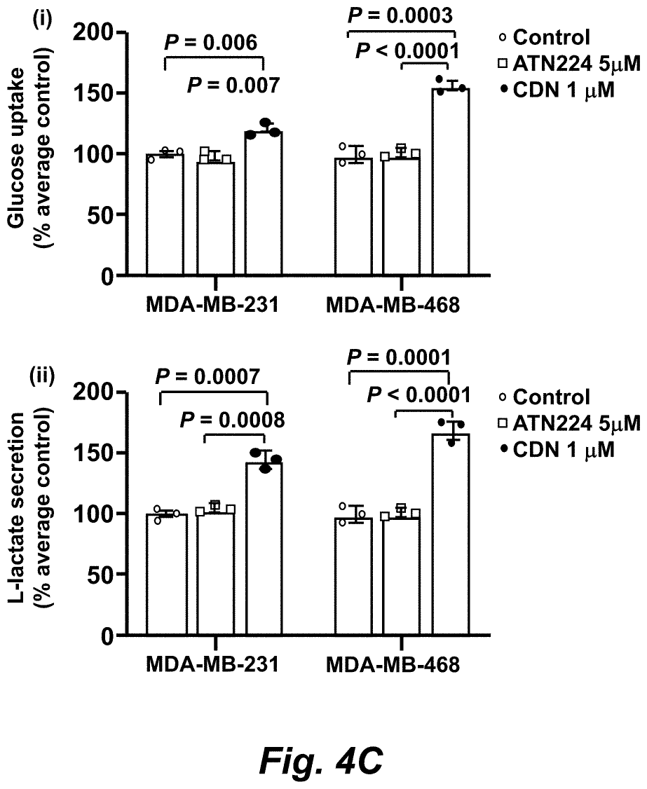

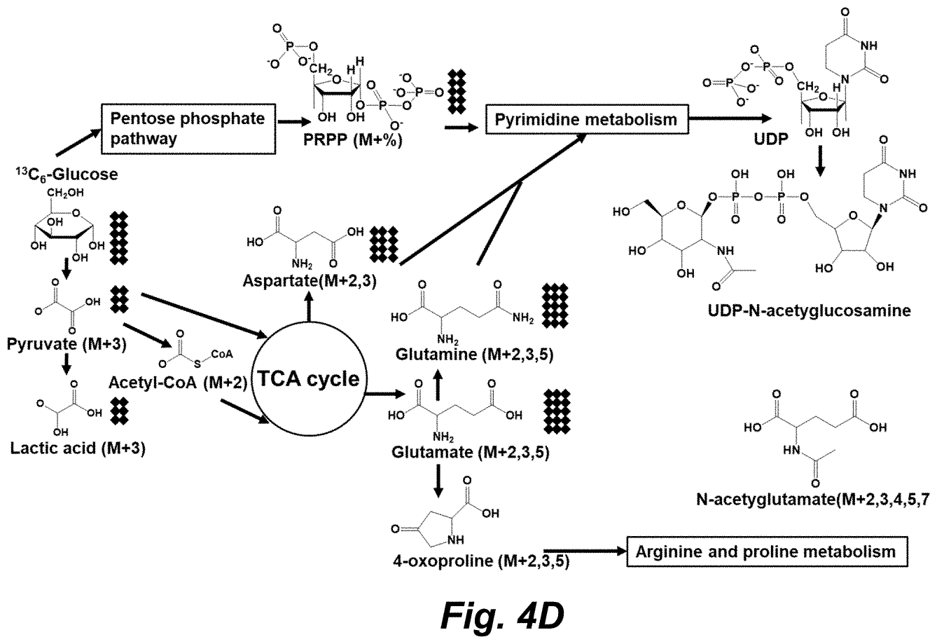

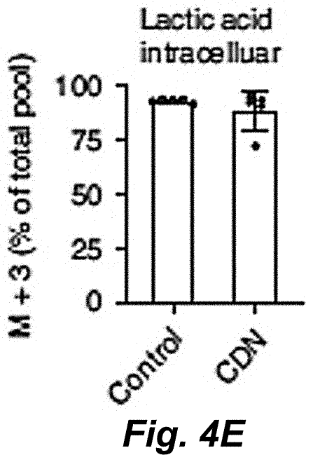

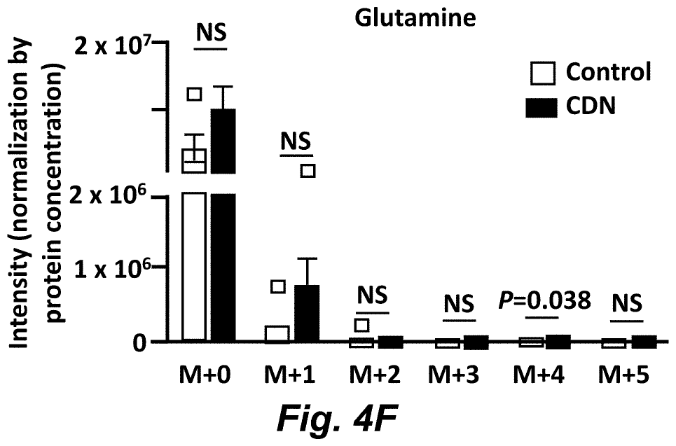

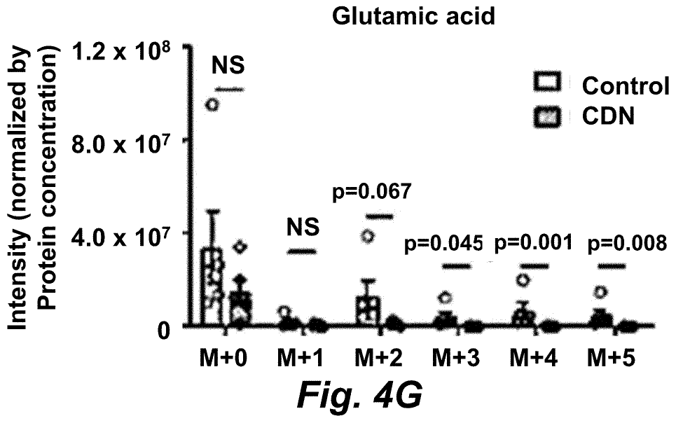

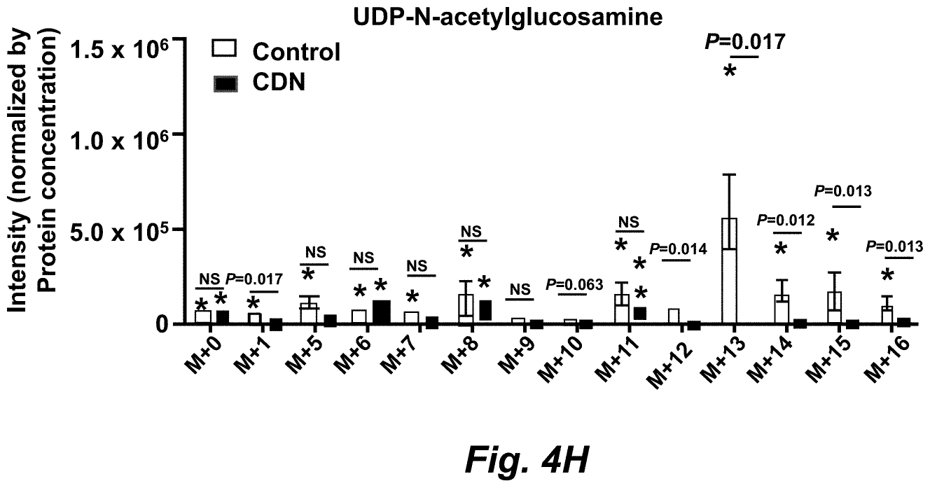

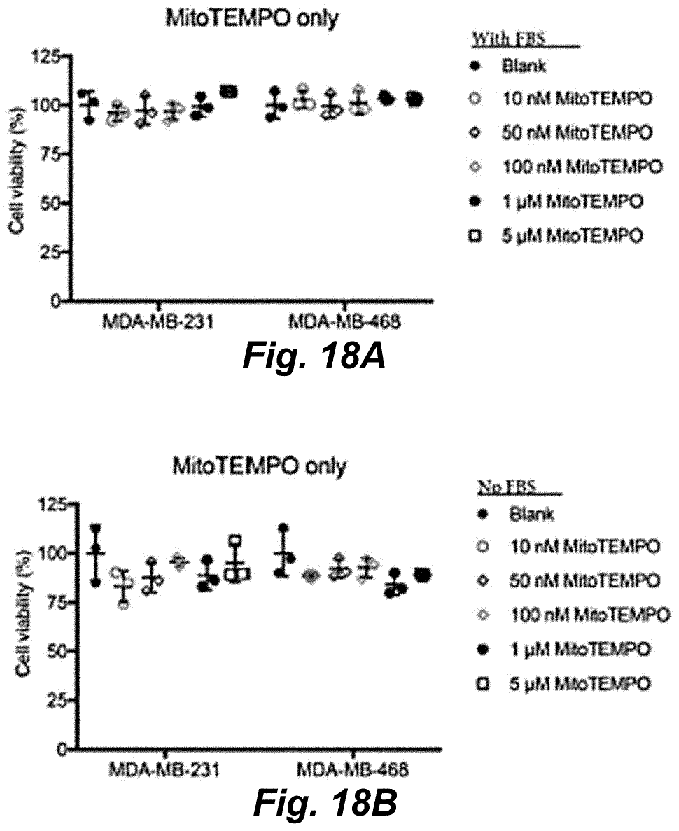

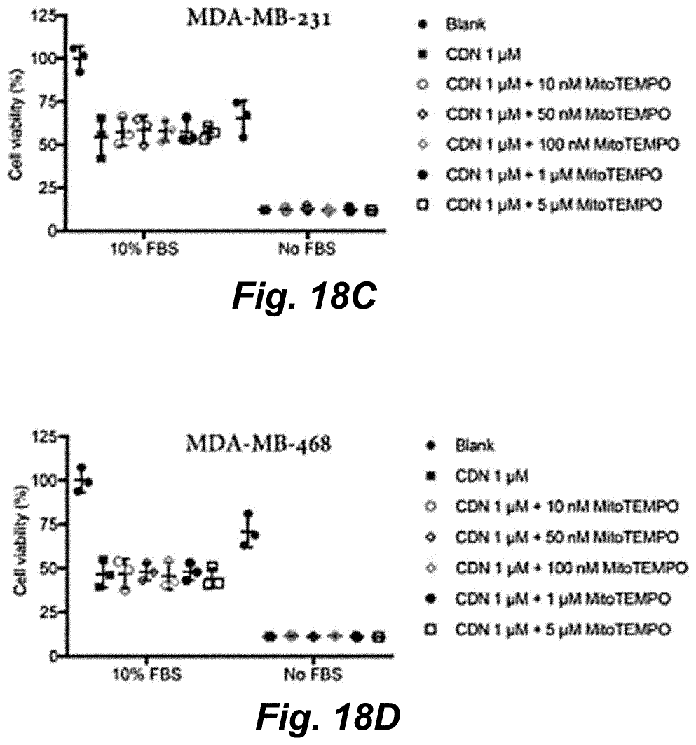

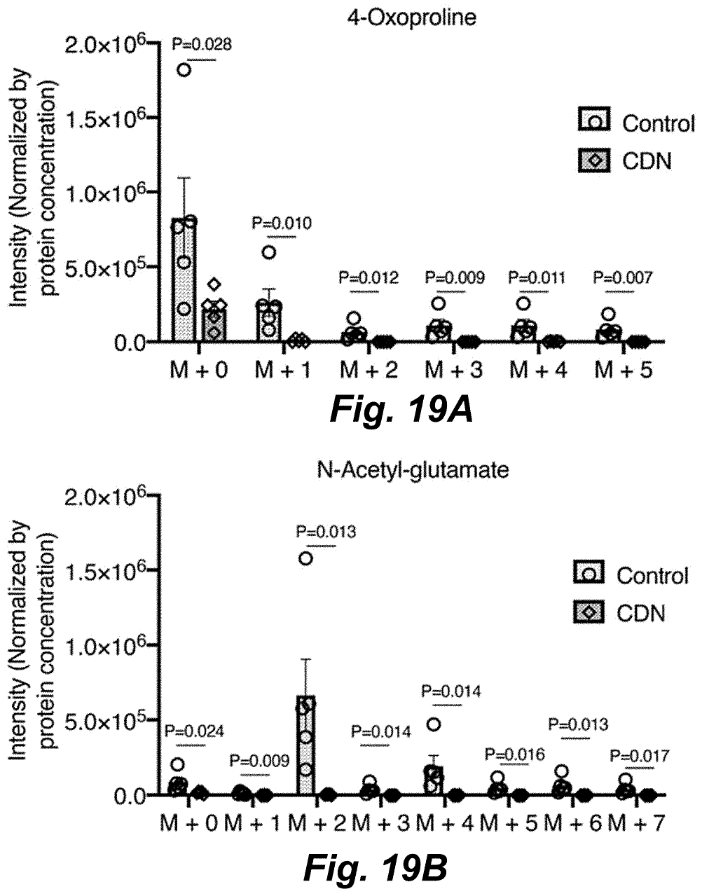

Cell redox balance correlates with cellular copper: Superoxide dismutase 1 (SOD1) uses copper as cofactor. Whether oxidative stress induced by copper depletion contributed to cell death was investigated. For both MDA-MB-231 and MDA-MB-468 cells, while SOD1 levels remained unaffected, SOD1 activity decreased after CDN treatment, showing a similar trend to that in treatment with a higher concentration of ATN224. The superoxide level in cells was quantified with a chemiluminescent tracer 2-methyl-6-phenyl-3,7-dihydroimidazo[1,2-a]pyrazin-3-one (CLA) (Yasuta et al., (1999) Chem. Lett. 28: 451-452) and was elevated threefold compared with non-treated controls as observed in both cell lines. This elevated oxidative stress led to DNA double-strand breaks (marked by γH2A.X) and detectable levels of lipid peroxidation (marked by 4-HNE) with immunofluorescence staining. However, the addition of mitochondrial superoxide scavenger MitoTEMPO with a concentration up to 5 μM did not rescue the cells from the CDN treatment effect (FIG. 18A-18D). These findings suggest that oxidative stress is not the direct cause of cell death but may exacerbate the cellular stress.
Inhibition of OXPHOS by CDN alters the metabolism of TNBC cells: After treatment with CDN, the OXPHOS was significantly inhibited and TNBC cells underwent a metabolic switch from OXPHOS to glycolysis. As shown in FIGS. 4A and 4B, together with inhibited OCR, ECAR was elevated in both MDA-MB-231 and MDA-MB-468 cells after 24 h of treatment with CDN, but not with other control agents. The ECAR levels were not responsive to the following injection of oligomycin, suggesting that cells have an increased glycolytic flux when OXPHOS is inhibited. Glucose uptake increased in both cell lines after 24 h of treatment with CDN (CDM, 1 μM), but not with ATN224 (5 μM) (FIG. 4C(i)). Extracellular lactate secretion significantly increased after CDN treatment (FIG. 4C(ii)), and intracellular lactate amount was comparable between CDN-treated and control cells (FIGS. 4D and 4E), suggesting increased flux to glycolysis.

Stable isotope-tracing experiments using 13C₆-glucose confirmed that CDN incubation led to a reduction of amino acid synthesis activity (FIGS. 4D, 4F, 4G, and 19A-19B). CDN treatment also caused changes in the hexosamine pathway, as evidenced by a significant decrease in UDP-N-acetylglucosamine (FIG. 4H). Stable isotope-tracing ¹³C ¹⁵N-glutamine also found significantly for amino acid and hexosamine synthesis and redox homeostasis, which all contributed to the potency of CDN treatment.

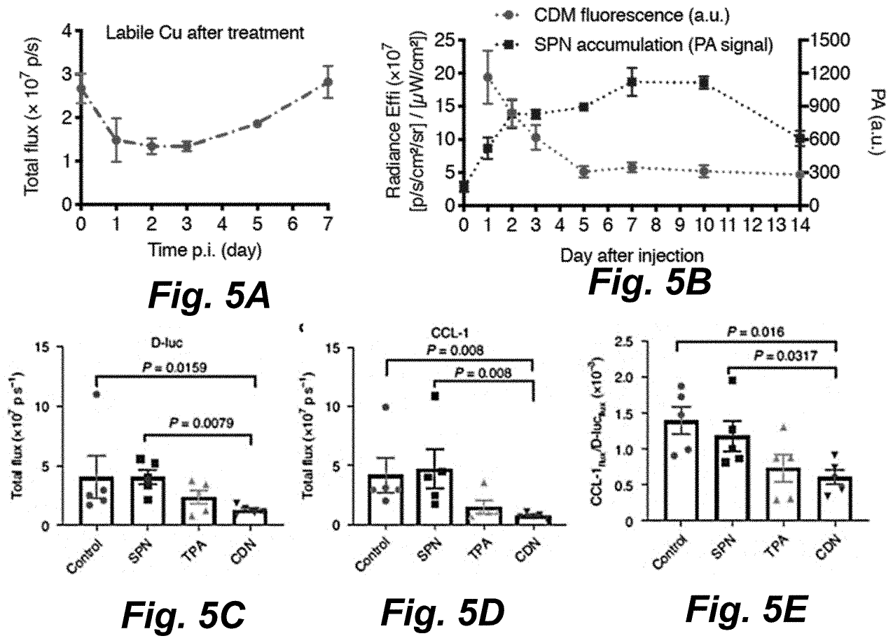

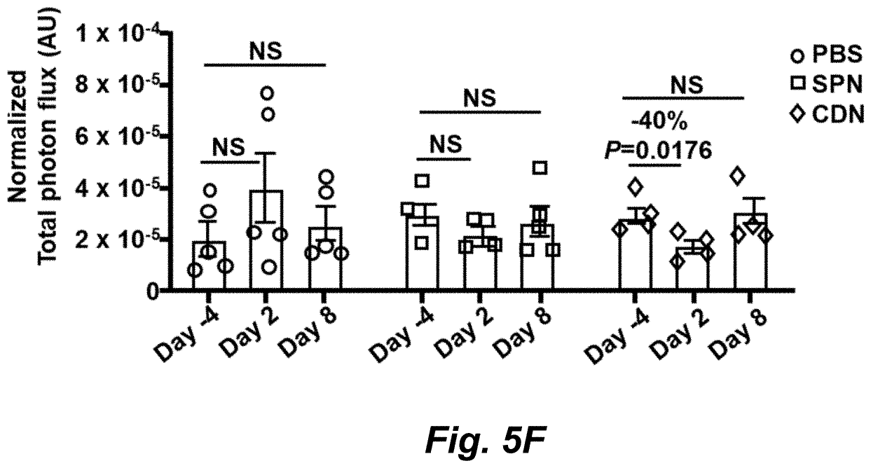

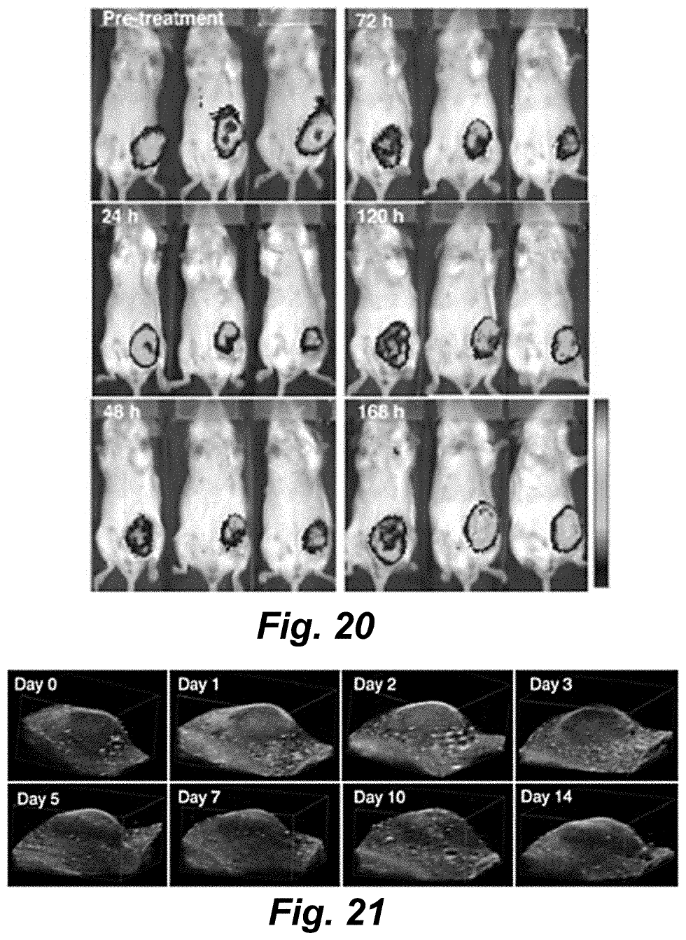

In vivo imaging of copper depletion and disruption of mitochondrial potential in the tumor by CDN: To study the copper-depleting dynamics in vivo, CCL-1 bioluminescence, CDM fluorescence, and SPN photoacoustic signals were longitudinally monitored in MDA-MB-231luc tumors after CDN administration. The labile copper level depicted by CCL-1 showed a continued decrease till day 3, and slowly resumed to the original level at day 7 after a single injection of CDN (CDM, 1.35 mg kg⁻¹) (FIGS. 5A and 20). While the nanoparticles were retained in the tumor region for as long as 10 d (FIGS. 5B and 21), CDM binding to copper was saturated around day 5, which explains the slow restoration of copper levels in tumors (FIG. 5B). These data suggest that CDN can accumulate in the tumor region, deplete copper locally and maintain a low copper level in the tumor for up to 5 d after a single injection (CDM, 1.35 mg kg⁻¹).

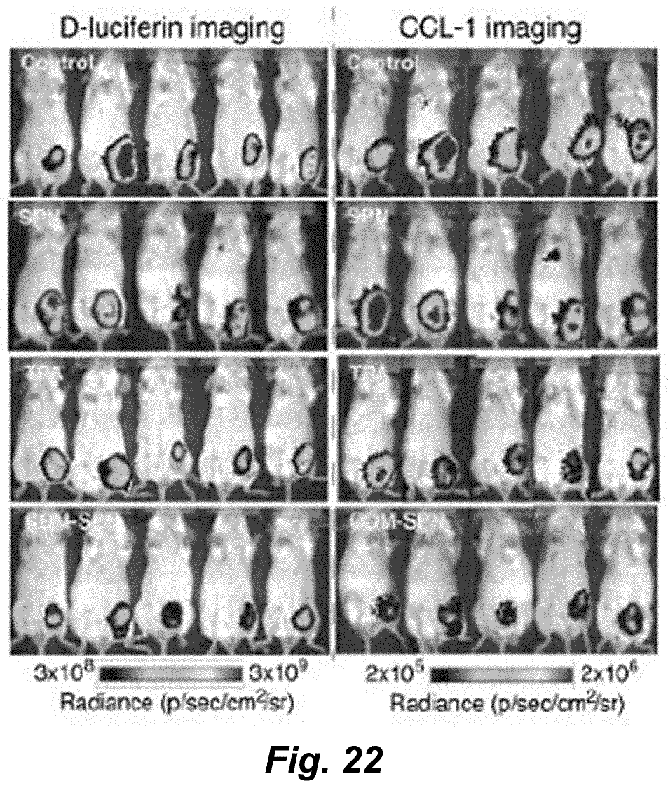

Whether strategic administration of CDN led to effective copper depletion and inhibition of tumor growth was investigated in MDA-MB-231luc orthotopic breast cancer-bearing mice (n=5 each group). For the treatment groups, mice received intravenous (i.v.) injection of CDN or TPA (at the same chelator dose of 1.35 mg kg⁻¹) every 3 d. On day 25 from the first treatment, mice were subjected to d-luciferin and CCL-1 bioluminescence imaging (FIG. 22). For both d-luciferin and CCL-1, the total photon flux values of the CDN group were significantly lower than the blank control and SPN control (FIGS. 5C and 5D). To normalize the cancer cell number effect on the CCL-1 imaging result, the total flux ratio of CCL-1 to d-luciferin was calculated (FIG. 5E). Both TPA and CDN successfully depleted the copper in the tumor region compared with the control group (TPA, P=0.04; CDN, P=0.016). However, CDN exhibited higher depletion efficiency and better treatment efficacy than TPA.

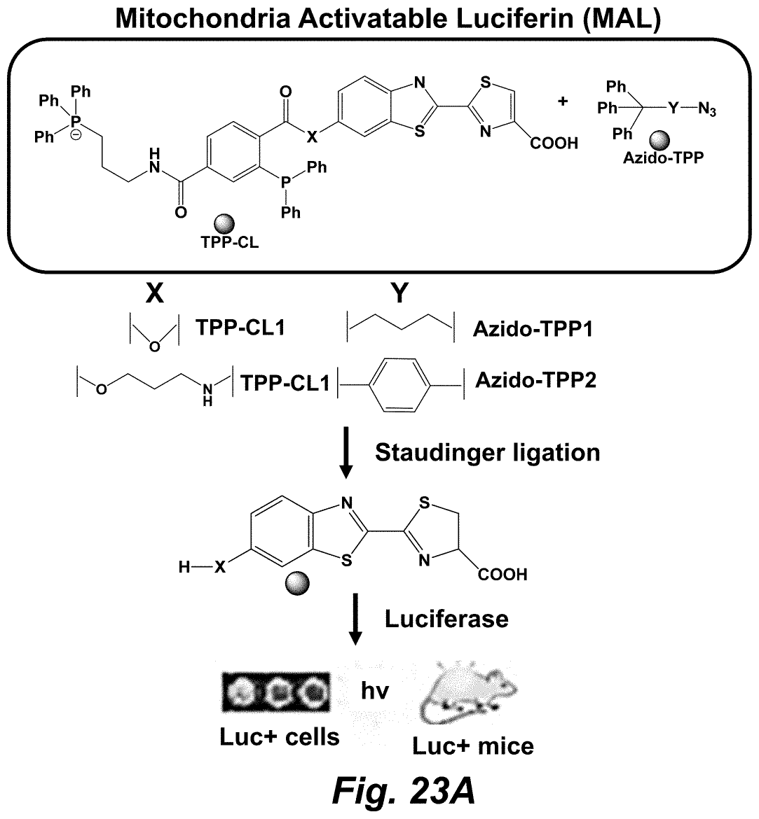

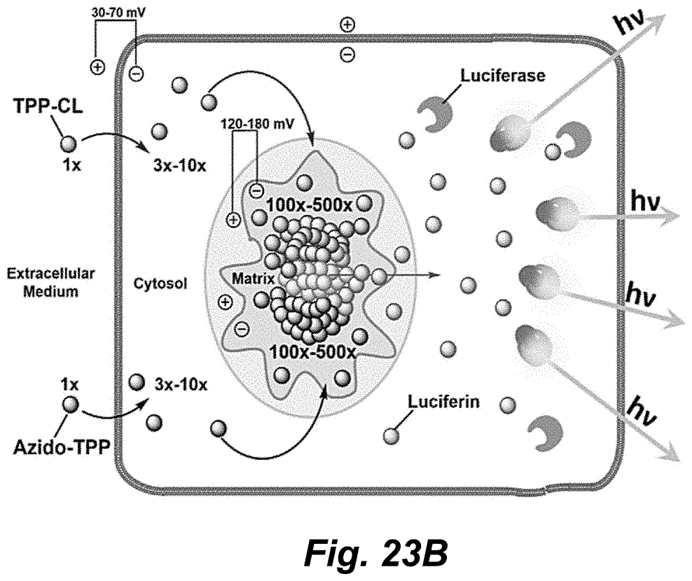

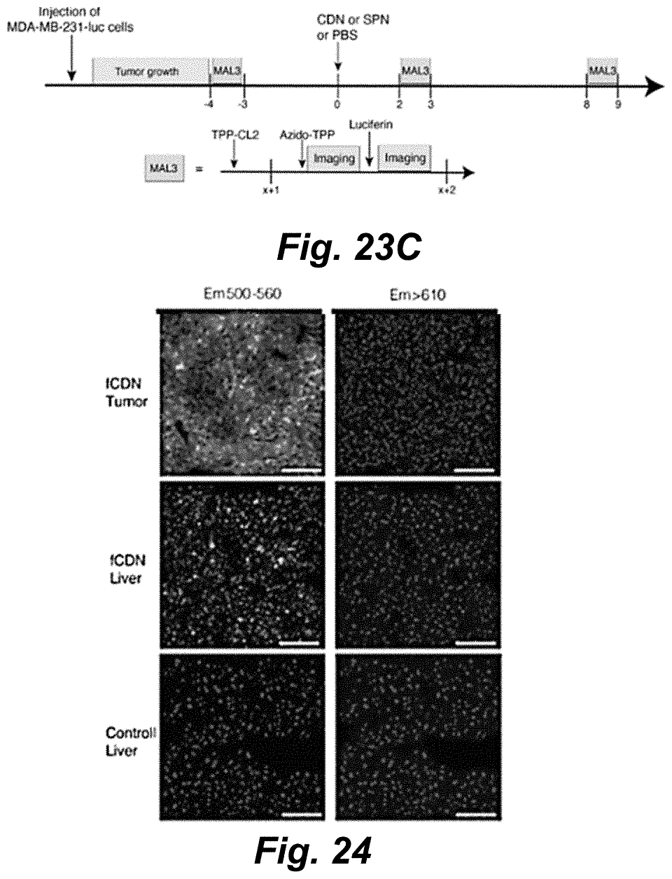

The mitochondrial membrane potential after CDN treatment in vivo was investigated using a mitochondria-activatable luciferin 3 (MAL3) probe (FIGS. 23A-23C). The mitochondrial membrane potential significantly decreased at 2 d after CDN treatment (P=0.0176, n=5), then restored to the pretreatment level when imaged at day 8 after treatment (FIG. 5F). In comparison, PBS or SPN treatment did not cause a significant change in the mitochondrial membrane potential. This result was consistent with in vitro findings that CDN treatment compromised mitochondrial membrane potential, which contributed to the apoptosis of cancer cells.

CDN serves as an effective, safe drug for TNBC: A potential side effect of systemic copper depletion is that it may result in overall copper deficiency and induce hematological disorders. We explored the biodistribution and copper depletion of the CDN in tumors and various healthy organs with fCDN. At 24 h after i.v. injection of fCDN, mice were killed, and the tumor and normal organs, including contralateral mammary fat pad, heart, lung, liver, spleen, adrenals, kidneys, stomach, small intestine, big intestine, ovary and uterus, were collected and imaged with an IVIS imager. The tumor showed significantly higher SPN fluorescence signal (green color) than any other healthy organs, whereas the CDM fluorescence at 740 nm (red color) was weak compared with that of the liver. Quantitatively, the tumor showed the highest fluorescence radiance ratio of 40.5±7.3, reflecting high accumulation and effective copper binding in the tumor. The liver also showed a relatively high amount of CDN as indicated by the CDM fluorescence, but the ratio of fluorescence emission at 540 nm to that at 740 nm (Em540/Em740) was only 2.5±0.9 for the liver. This indicated that although CDNs were cleared through the hepatobiliary route, they did not deprive the liver of copper ions. Distinct distribution patterns inside the tumor and liver tissues have been observed with microscopic imaging (FIG. 24). CDN signals in the liver appeared punctuated and showed a good correlation between green and red signals (low copper binding). In the tumor, the green signals were relatively homogenously distributed and red signals were rather dim (due to high copper binding). CDN likely resided in the Kupffer cells in the liver instead of hepatocytes. Other healthy major organs showed minimal fluorescence signals in both channels, indicating fairly low uptakes in normal tissues.

The potential cumulative toxicity originating from repetitive injections and acute toxicity was evaluated. For the cumulative toxicity, healthy Balb/c mice received weekly i.v. administration of CDN. During the treatment, no weight loss or behavioral abnormality was observed. After seven injections of CDN, mice were killed for hematological and pathological analysis. No blood count suppression was observed in the treated group and no difference was observed in the liver panel comparing with the saline control group. No pathological changes were observed in major organs, including the hearts, lungs, livers, kidneys and spleens. For the acute toxicity study, healthy mice were intravenously injected with 100 mg kg⁻¹ CDN or saline and monitored for 2 weeks. High-dose injection did not cause either body weight loss or behavioral abnormality in the following 2 weeks, or hematological or pathological changes compared with the control group.

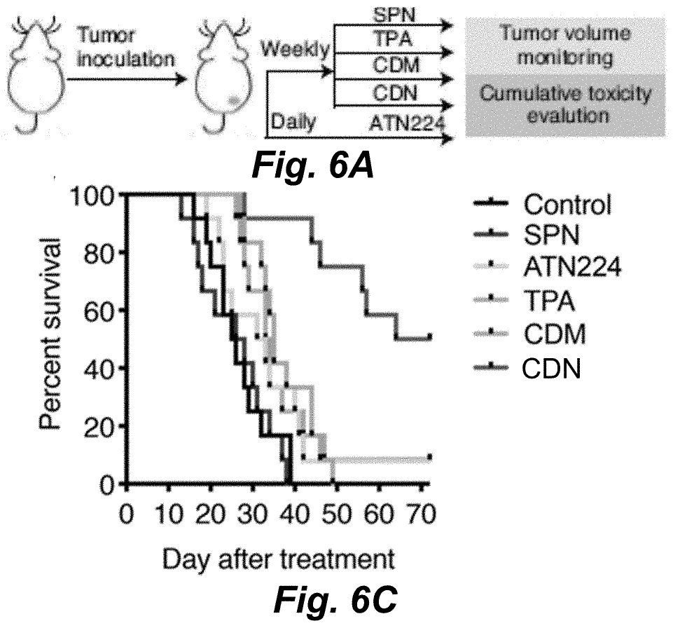

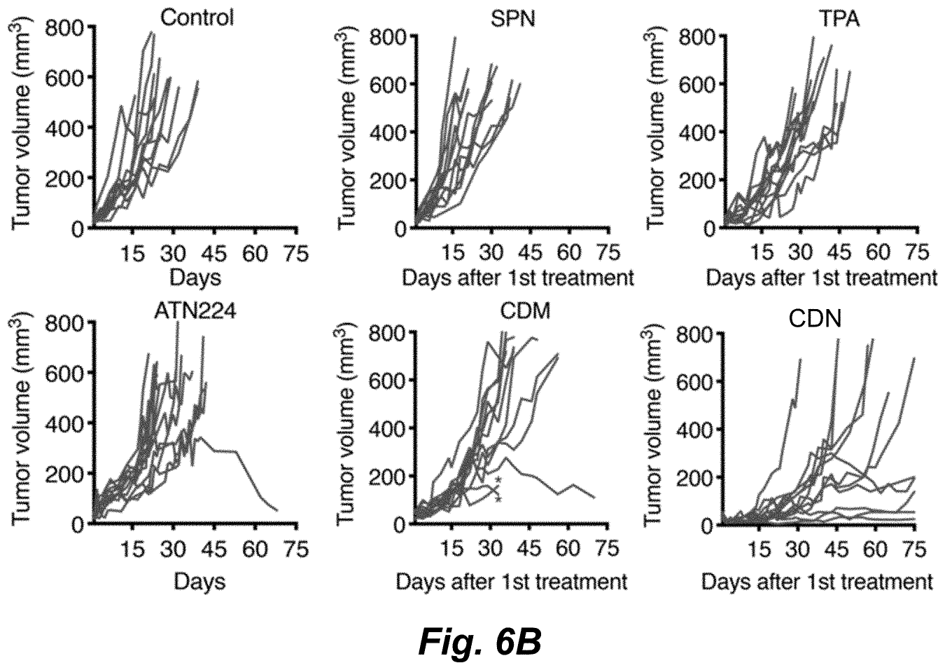

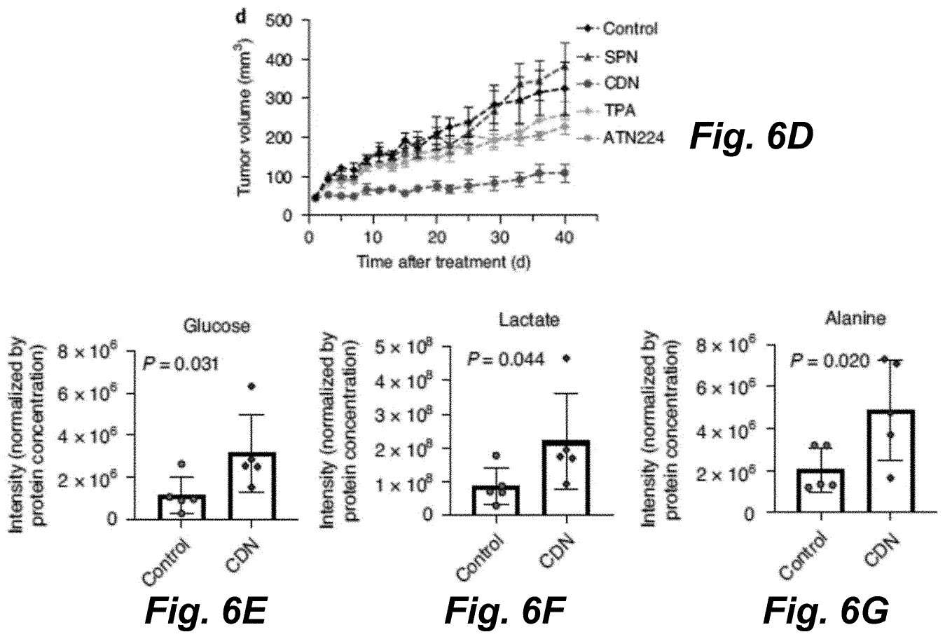

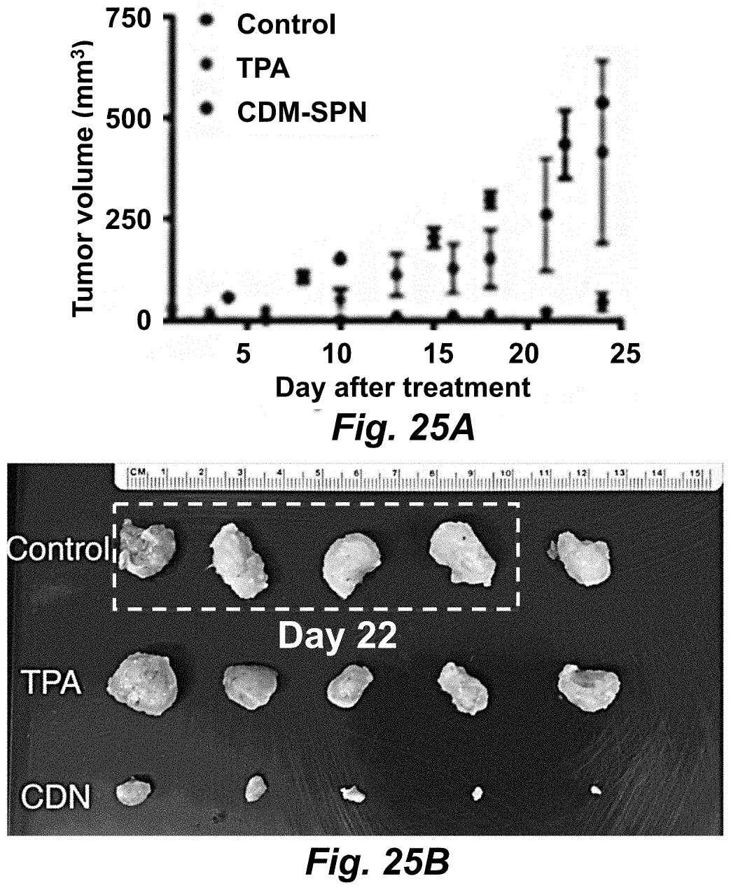

The therapeutic effect of CDN for TNBC was first evaluated in a long-term survival study in the MDA-MB-231 breast cancer model. For the treatment groups, nude mice bearing MDA-MB-231 orthotopic tumors received seven doses of i.v. injections of TPA, CDM or CDN (chelator dose of 1.35 mg kg⁻¹, n=12 each group) or daily oral gavage of ATN224 (0.7 mg kg⁻¹ d⁻¹ until day 42, n=12). Since the copper level in the tumor resumed to the original level at day 7 after a single injection (FIG. 5A), a weekly dosing strategy was adopted to explore if a lower overall dosing was applicable (FIG. 6A). As shown in FIGS. 6B and 6C, tumor growth in the saline group and the SPN group was rapid, and the median survival was 25.5 d and 27 d, respectively. The ATN224 treatment group showed moderate therapeutic efficacy with a median survival of 32 d. The TPA and CDM groups showed similar therapeutic efficacy (median survival, 33.5 d for CDM and 35 d for TPA). The CDN treatment significantly inhibited tumor progression: 50% of the treated mice survived over 68 d after the first injection, and the tumor volumes of the surviving mice remained fairly small except for one mouse (mean±s.e.m., 99±29 mm3, n=5). This promising therapeutic efficacy of CDN for primary TNBC tumors was also observed in mouse models bearing MDA-MB-468 and 4T1 tumors (FIGS. 6D, 25A, and 25B). Metabolomic analysis of the tumors further confirmed that CDN treatment truly led to elevated glycolysis, evidenced by significantly higher levels of glucose (FIG. 6E), glycolysis end product lactate (FIG. 6F) and alanine, the product of pyruvate via transaminase reactions (FIG. 6G).

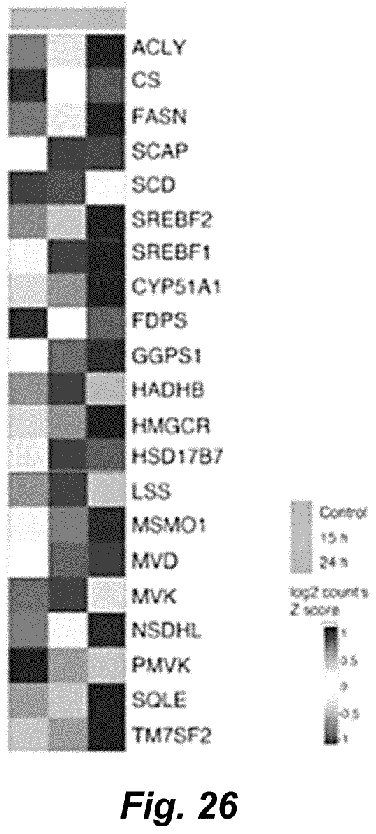

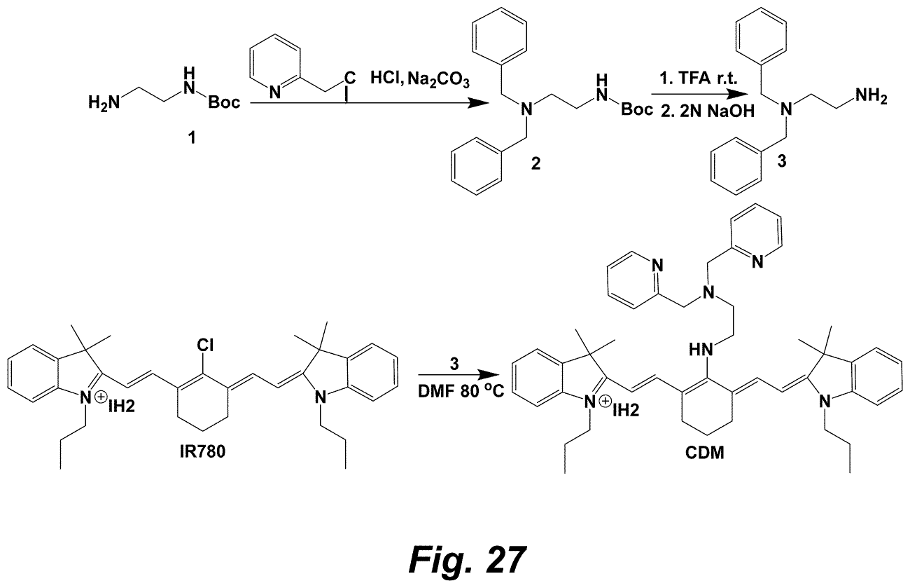

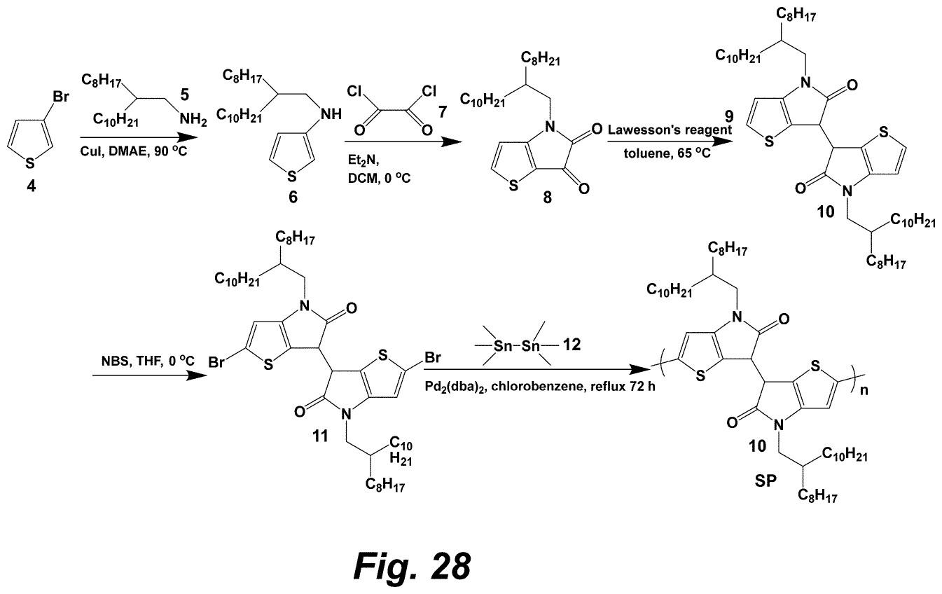

The mRNA sequencing results showed that lipid metabolism was likely altered in response to copper depletion. TNBC typically shows upregulation of genes encoding enzymes for fatty acid oxidation and downregulation of genes encoding enzymes for fatty acid synthesis in primary human tumors. After CDN treatment, MDA-MB-231 cells showed upregulation of many genes that encode activators of fatty acid, cholesterol and triglyceride synthesis, including ACLY, FASN, SCAP, SREBF1 and SREBF2 (FIG. 26). Enhanced fatty acid synthesis may further exacerbate the energy deficiency as increased malonyl-CoA inhibits carnitine palmitoyl transferase I (CPT1) and suppresses fatty acid transport into the mitochondria for oxidation (McGarry et al., (1977) J. Clin. Invest. 60: 265-270; McGarry et al., (1978) J. Biol. Chem. 253: 4128-4136).

Copper enzymes including COX (Ackerman et al., (2018) J. Biol. Chem. 293: 4628-4635) or phosphodiesterase 3B (Krishnamoorthy et al., (2016) Nat. Chem. Biol. 12: 586-592) have been found to be excessive and essential for cancer cell metabolism, and various others have been identified as prerequisite during all stages of cancer development, involving uncontrolled cancer cell proliferation, dysregulated metabolism, invasion and migration to distant sites (Ishida et al., (2013) Proc. Natl Acad. Sci. USA 110: 19507-19512; Turski et al., (2009) J. Biol. Chem. 284, 717-721; Blockhuys & Wittung-Stafshede (2017) Int. J. Mol. Sci. 18: 871; Denoyer et al., (2015) Metallomics 7: 1459-1476; Hanahan & Weinberg (2011) Cell 144: 646-674; Grubman & White (2014) Expert Rev. Mol. Med. 16: e11; Matson Dzebo et al., (2016) Biomol. Concepts 7: 29-39). Despite the recognized importance of copper enzymes, successful attempts to treat primary or metastatic cancer with copper chelation are rather limited (Shao et al., (2017) Nat. Biomed. Eng. 1: 745-757). Conventional cop-per chelator TM and ATN224 are reported to be anti-angiogenesis agents (Pan et al., (2002) Cancer Res. 62: 4854-4859; Donate et al., (2008) Br. J. Cancer 98: 776-783). The cytotoxicity of TM and ATN224 to TNBC cells is 100 μM) (Chisholm et al., (2016) Oncotarget. 7, 84439-84452). Also, their therapeutic effect in primary tumor treatment is not satisfactory for many types of cancer (Lin et al., (2013) Urol. Oncol. Semin. Orig. Investig. 31: 581-588; Redman et al., (2003) Clin. Cancer Res. 9: 1666-1672; Schneider et al., (2013) New Drugs 31: 435-442). The most successful case so far was seen in a phase II clinical trial with TM as a chemo-preventive method in prolonging the recurrence-free window of patients with no trace of tumor by the time of the treatment (Garber, K., (2015) Science 349: 128-129; Chan et al., (2017) Clin. Cancer Res. 23: 666-676; Sahota et al., (2017) J. Clin. Oncol. 35: 2557-2557). Moreover, both molecules deplete copper systemically, and serious concerns have been raised that this may result in over-all copper deficiency and induce hematological disorder and neurotoxicity. Functioning differently than these conventional copper chelators, CDN induced mitochondrial dysfunction and altered the metabolism of TNBC cells. Copper depletion by CDN resulted in a combination of energy and nutrient deficiency, as well as elevated oxidative stress and mitochondrial membrane rupture, which all contributed to the apoptosis of TNBC cells. The design and application of CDN may open new avenues for copper-depleting cancer intervention.

One aspect of the disclosure encompasses embodiments of a nanoparticle comprising a self-reporting copper-depleting moiety (CDM) and a matrix, wherein the matrix comprises a semi-conducting polymer and a phospholipid-polyethylene glycol (PEG), wherein the CDM is embedded in or on the matrix.

In some embodiments of this aspect of the disclosure, the copper-depleting moiety (CDM) can be linked to a fluorescent moiety.

In some embodiments of this aspect of the disclosure, the copper-depleting moiety (CDM) can be N,N-bis(2-pyridinylmethyl)-1,2-ethanediamine.

In some embodiments of this aspect of the disclosure, the fluorescent moiety can be a tricarbocyanine.

In some embodiments of this aspect of the disclosure, the linkage of the CDM to the fluorescent moiety can be configured to allow the CDM, when bound to a copper ion, to quench a fluorescence emission from the fluorescent moiety.

In some embodiments of this aspect of the disclosure, the semi-conducting polymer can be a photoacoustic semi-conducting polymer.

Another aspect of the disclosure encompasses embodiments of a method of reducing the amount of free copper ions in the mitochondria of a target cell or population of target cells, the method comprising the step of delivering a nanoparticle to the interior of a target cell, wherein the nanoparticle comprises (a) a matrix comprising a semi-conducting polymer and a phospholipid-polyethylene glycol (PEG); and (b) a self-reporting copper chelator (CDM) embedded in or on the matrix, whereby copper ions in the mitochondria of the recipient cell chelate to the nanoparticle, thereby reducing the level of free copper in the mitochondria.

In some embodiments of this aspect of the disclosure, the copper-depleting moiety (CDM) can be linked to a fluorescent moiety, wherein the linkage of the CDM to the fluorescent moiety can be configured to allow the CDM, when bound to a copper ion, to quench a fluorescence emission from the fluorescent moiety.

In some embodiments of this aspect of the disclosure, the copper-depleting moiety (CDM) can be N,N-bis(2-pyridinylmethyl)-1,2-ethanediamine.

In some embodiments of this aspect of the disclosure, the fluorescent moiety can be a tricarbocyanine.

In some embodiments of this aspect of the disclosure, the semi-conducting polymer can be a photoacoustic semi-conducting polymer.

In some embodiments of this aspect of the disclosure, the target cell can be a cancer cell and the population of target cells can be in a tumor.

In some embodiments of this aspect of the disclosure, the target cell or the population of target cells can be in an animal or human subject tumor.

In some embodiments of this aspect of the disclosure, the method can further comprise the step of detecting a change in an optical signal generated from the nanoparticle by chelation of copper ions to the nanoparticle.

In some embodiments of this aspect of the disclosure, the method can further comprise the step of detecting a photoacoustic signal from the nanoparticle and determining the location of the nanoparticle within the animal or human subject.

Yet another aspect of the disclosure encompasses embodiments of a method of reducing at least one of the proliferation and the viability of a cancer cell by administering a self-reporting copper-depleting moiety (CDM) and a matrix, wherein the matrix comprises a semi-conducting polymer and a phospholipid-polyethylene glycol (PEG), wherein the CDM is embedded in or on the matrix, wherein copper ions in the mitochondria of the recipient cell are chelated by the CDM, thereby reducing the level of free copper in the mitochondria and reducing at least one of the proliferation and the viability of a cancer cell in the patient.

In some embodiments of this aspect of the disclosure, the method can further comprise copper-depleting moiety (CDM) is linked to a fluorescent moiety, wherein the linkage of the CDM to the fluorescent moiety is configured to allow the CDM, when bound to a copper ion, to quench a fluorescence emission from the fluorescent moiety, wherein the copper-depleting moiety (CDM) is N,N-bis(2-pyridinylmethyl)-1,2-ethanediamine, the fluorescent moiety is a tricarbocyanine and the semi-conducting polymer is a photoacoustic semi-conducting polymer.

In some embodiments of this aspect of the disclosure, the cancer can be Triple Negative Breast Cancer (TNBC).

In some embodiments of this aspect of the disclosure, the method can further comprise the step of detecting at least one of an optical signal from the nanoparticle generated after chelation of copper ions to the nanoparticle and detecting the presence of the cancer in the patient by detecting a change in the optical signal from the nanoparticle generated by chelation of copper ions to the nanoparticle and determining the location of the cancer in the patient.

In some embodiments of this aspect of the disclosure, the method can further comprise adjusting the level of a dose of a therapeutic agent administered to the patient in need thereof, whereby the change of the intensity of the optical signal indicates the level of the reduction of the proliferation or viability of the cancer cell caused by the therapeutic agent

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

### Example 1

Chemicals: All chemicals were obtained from Sigma-Aldrich unless otherwise stated. 1,2-dipalmitoyl-sn-glycero-3-phosphoethanolamine-N-[methoxy(polyethyleneglycol)-2000](ammonium salt) (DPPE-PEG2000) was from Avanti Polar Lipids. Poly[(9,9-dioctyl-2,7-divinylenefluorenylene)-alt-{2-methoxy-5-(2-ethylhexyloxy)-1,4-phenylene}] (fSP) was from Luminescence Technology. CLA was from TCI America. JC-1 dye was from Invitrogen. ATN224 was from Cayman Chemical Company. XenoLight d-Luciferin was from PerkinElmer. TRIzol reagent was from Thermo Fisher Scientific. Ultrapure water was from Invitrogen. 1×PBS, 1×D-PBS and 1×HBSS were from Gibco. Sterile 0.9% saline solution was from TEKnova. CCL-1 was synthesized with the procedures reported previously (Heffern et al., (2016) Proc. Natl Acad. Sci. U.S.A. 113:14219-14224).

### Example 2

Compound synthesis and characterization: NMR spectra were taken on a Bruker 400 MHz magnetic resonance spectrometer. Chemical shifts are reported as 6 in units of p.p.m. relative to TMS (δ 0, s); multiplicities are reported as follows: s (singlet), d (doublet), m (multiplet) or br (broadened); coupling constants are reported as a J value in hertz (Hz); the number of protons (n) for a given resonance is indicated as nH, and based on the spectral integration values. Elemental analysis was performed on a FlashEA1112 Elemental Analysis Instrument. Gel permeation chromatography analysis was conducted on a Waters 2414 system with polystyrene as standard and chloroform as eluent. UV-visible-near-infrared absorption spectra were recorded on an Agilent spectrophotometer.

### Example 3

CDN formulation and characterization: CDN and fCDN were formulated with a nano-emulsion method reported previously (Pu et al. (2014) Nat. Nanotechnol. 9: 233-239). Briefly, 1 ml of tetrahydrofuran solution of semiconducting polymer (SP) or fSP (0.125 mg), DPPE-PEG2000 (2.5 mg) and CDM (2.5 μg) was rapidly injected into distilled deionized water (9 ml) under continuous sonication with a microtip-equipped probe sonicator (Branson, W-150) at a power output of 6 W root mean square (RMS) for 2 min. After sonication, tetrahydrofuran was evaporated at 45° C. under nitrogen atmosphere. The aqueous solution was filtered through a polyvinylidene difluoride syringe-driven filter (0.22 μm) (Millipore) to remove large nanoparticles. Then the solution was washed three times with HEPES buffer (10 mM, pH 7.4) with a 30-kDa centrifugal filter unit (Millipore) under centrifugation at 3,500 r.p.m. for 8 min at 4° C. After ultrafiltration, the final concentration of CDM in CDN was 132 μM (total nanoparticle matrix concentration was 6.5 mg ml-1). The concentrations of diluted CDN solution were presented in the amount of encapsulated CDM. CDN solution was stored in the dark at 4° C. The sizes of CDN and fCDN were determined by DLS (Malvern ZetaSizer Nano S) and presented as number percentage and intensity percentage with polydisperse index. The TEM images were obtained on a transmission JEM 1230 electron microscope with an accelerating voltage of 200 kV. Zeta potential measurements were conducted on the Malvern ZetaSizer Nano S with folded capillary cells. Fluorescence spectra were recorded on a wavelength-calibrated FluoroMax-3 fluorometer (Horiba Jobin Yvon).

### Example 4

Fluorescence response of CDN to copper and other ions: To measure the fluorescence signal changes of CDN upon mixing with Cu⁺ and Cu²⁺, different equivalents of copper ions were added to CDN solution (CDM, 1 μM), and the fluorescence emission spectra were read on a 96-well plate reader (Tecan). For fCDN, the fluorescence spectra were measured upon mixing with different equivalents of Cu²⁺ with an excitation wavelength at 453 nm. The FRET ratio was calculated as the ratio between emission at 540 nm and 740 nm.

To test the specificity of CDN, CDN solution (CDM, 5 μM) was first mixed with physiologically relevant metal ions, including Na⁺ (5 mM), Mg²⁺ (5 mM), K⁺ (5 mM), Ca²⁺ (5 mM), Zn²⁺ (100 μM), Fe²⁺ (20 μM), Mn²⁺ (1 μM), Ni²⁺ (0.5 μM), Cd²⁺ (0.5 μM) and Co²⁺ (0.5 μM). Concentrations of the tested metal ions are selected based on the physiological abundancy. The fluorescence emissions of CDM after mixing with metal ions were measured (excitation (Ex), 610 nm; emission (Em), 740 nm). Then, an extra 5 μM of Cu²⁺ was added to each group of mixtures to determine the CDM fluorescence change. For the phantom imaging, agar phantom, CDN solutions with different equivalents of Cu⁺ to CDM were added to warm agar solution (1%, 20 μl, 40° C.) and immediately added to a preformulated agar phantom well. Each well was sealed with another layer of agar. The fluorescence image was acquired with IVIS Spectrum (Ex, 640 nm; Em, 740 nm), and the photoacoustic signal of each well was acquired with LAZR VisualSonics (1,064 nm).

### Example 5

Serum stability of CDN: First, 4 μl of fCDN was added to 396 μl of mouse serum (Sigma-Aldrich) without or with 500 μM EDTA for labile copper removal, and incubated for 24 h at 37° C. The FRET ratios between 540 nm and 740 nm (Ex, 453 nm) and CDM fluorescence (Ex, 610 nm; Em, 740 nm) before or after incubation were measured with a plate reader (Tecan). To determine the CDM leakage in serum over 24 h, CDN in serum after incubation was washed with an ultracentrifugation unit (Amicon Ultra-0.5 ml). The CDM amount in the elute was determined by fluorescence measurement. To determine the effect of CDN on serum copper, CDN (CDM, 2 μM or 4 μM) was added to serum and incubated for 24 h at 37° C. The ceruloplasmin activity in the serum was determined by Ceruloplasmin Colorimetric Activity Kit (Invitrogen, EIACPLC) under the sample preparation guidelines and assay protocol provided. Results were presented as ratio percentage to the ceruloplasmin activity (mU ml⁻¹) before incubation.

### Example 6

Cell culture: MDA-MB-231, MDA-MB-468, BT-20, 4T1, WI-38, MCF-10A, RWPE-1, PC-3, 22Rv1, HCC1428, MCF7 and T47D were from ATCC. MDA-MB-231luc cells were from Promega. Cells were cultured in a humidified atmosphere of 5% CO₂ and 95% air at 37° C. unless otherwise stated. MDA-MB-231, MDA-MB-231luc, MDA-MB-468, 4T1, 4T1luc, 22Rv1 and HCC1428 cells were cultured in RPMI-1640 medium (Gibco, cat. no. 22400089) with 10% FBS and 1% pen/strep. MCF7 cells were cultured in ATCC-formulated Eagle's MEM (ATCC, cat. no. 30-2003) supplemented with 0.01 mg ml⁻¹ human recombinant insulin (Gibco, cat. no. 12585-014), 10% FBS and 1% pen/strep. T47D cells were cultured in RPMI-1640 medium (Gibco, cat. no. 22400089) supplemented with 0.2 U ml⁻¹ human recombinant insulin (Gibco, cat. no. 12585-014), 10% FBS and 1% pen/strep. BT-20 and WI-38 cells were cultured in ATCC-formulated Eagle's MEM (ATCC, cat. no. 30-2003) with 10% FBS and 1% pen/strep. PC-3 cells were cultured in ATCC-formulated F-12K medium (ATCC, cat. no. 30-2004) with 10% FBS and 1% pen/strep. MCF-10A cells were cultured in MEBM base medium with MEGM kit supplement (without the addition of gentamycin-amphotericin B mix, Lonza/Clonetics, cat. no. CC-3150), 100 ng ml⁻¹ cholera toxin (Sigma-Aldrich, cat. no. C8052) and 1% pen/strep. RWPE-1 cells were cultured in Keratinocyte Serum Free medium (Gibco, cat. no. 17005042) with K-SFM supplement, including EGF and BPE. Mycoplasma test was carried out routinely to detect contamination.

### Example 7

In vitro copper depletion: The copper level after CDN or control agent treatment was measured by CCL-1 luminescence imaging or ratiometric fluorescence microscopic imaging of fCDN. For CCL-1 imaging, MDA-MB-231luc or 4T1luc cells were seeded in six-well plates (10⁶ per well) 24 h before the treatment and cultured in a humidified atmosphere of 5% CO₂ and 95% air at 37° C. For MDA-MB-231luc cells, CDN (CDM, 1 μM) with or without extra copper ion supplements at concentrations indicated in FIG. 2 or 500 μM EDTA was added to each well (fresh medium with 10% FBS) and incubated for 24 h. For 4T1luc cells, CDN, SPN, ATN224 or TPA (chelator concentration, 1 μM; SPN, 50 μg ml-1) was added and incubation was continued for 24 h. Cells were washed three times with PBS, and 1 ml of CCL-1 in D-PBS (50 μM) was added to each well. Cells were immediately imaged by IVIS Spectrum (PerkinElmer) for 45 min. The integration images of luminescent radiance were presented. For ratiometric fluorescence imaging, MDA-MB-231 or MDA-MB-468 cells were seeded (2×10⁴ per well) in a Lab-Tek II chamber (NUNC) 24 h before the experiment and cultured in a humidified atmosphere of 5% CO₂ and 95% air at 37° C. After 24 h of incubation with fCDN or with supplements of either 10 μM Cu²⁺ or 500 μM EDTA, cells were washed three times with PBS and changed to fresh medium containing 10 μg ml⁻¹ Hoechst 33342. For live-cell confocal microscopic imaging (Zeiss LSM 710), 488-nm excitation wavelength was used and fluorescence signals at 525±30 nm were collected as the green channel (fSP) and at 630-733 nm were collected as the red channel (CDM). Images were processed as green/red ratios profiled by ImageJ and presented in pseudo-colors.

### Example 8

Cytotoxicity: For the MTS assay, TNBC and normal cells were seeded into 96-well plates 24 h before the test and cultured in a humidified atmosphere of 5% CO₂ and 95% air at 37° C. Cells were then incubated with CDN or control agents with the concentrations indicated in FIG. 2 in complete medium or base medium without FBS supplement. At 24 h after incubation, cells were washed with PBS three times and changed to fresh medium. The viability of the cells after treatment was measured using the MTS assay (CellTiter 96, Promega, cat. no. G3580). The viability was presented as the percentage of control (n=3 per condition for viability). For the trypan blue exclusion test, TNBC cells were seeded into six-well plates 24 h before the test and cultured in a humidified atmosphere of 5% CO₂ and 95% air at 37° C. Cells were then incubated with CDN or control agents in complete medium. At 24 h after incubation, cells were washed with PBS three times and collected with a scraper. The cell suspension was then mixed with 0.4% trypan blue dye (1:1; Thermo Fisher Scientific, cat. no. 15250061) and incubated for less than 3 min at room temperature. The viability of the cells was measured with Countess II (Life Technology). The viability was presented as the percentage of control (n=3 per condition for viability).

### Example 9

Cell invasion study: Cell suspension was added into the upper chambers of the prehydrated 24-well plate Matrigel invasion chamber (Corning, cat. no. 08-774-122) and allowed to grow for 24 h in complete medium with or without CDN or control agent treatment. After incubation, the non-invaded cells were removed with cotton swab scrubbing, and the invaded cells were stained with Diff-Quik. Cell numbers were counted with a microscope with at least 15 views per well. For MDA-MB-231 cells, n=3 biologically independent samples for blank, SPN, CDN with copper, TPA and ATN224 groups; n=5 biologically independent samples for the CDN group. For MDA-MB-468 cells, n=4 biologically independent samples for blank and SPN groups, and n=5 biologically independent samples for the CDN group.

### Example 10

Western blots: Cells were treated with CDN or control agents with the concentration indicated in FIG. 12. Then cells were trypsinized, and cell lysates were prepared with RIPA buffer (Thermo, cat. no. 89900). The protein concentrations were quantified by Pierce BCA assay (Thermo Scientific, 23250). Cleaved caspase 3 antibody was purchased from Cell Signaling Technologies (Asp175, no. 9661). SOD1 antibody was from Santa Cruz Biotechnologies (sc-101523).

### Example 11

In vitro mitochondrial membrane potential imaging: Mitochondrial membrane potential was characterized by JC-1 staining (Thermo Fisher, T3168). Briefly, cells were seeded (2×10⁴ per well) in a Lab-Tek II chamber (NUNC) 24 h before the treatment and cultured in a humidified atmosphere of 5% CO₂ and 95% air at 37° C. After 24 h of incubation with CDN (with or without 10 μM copper supplements) or control agents, cells were washed three times with PBS and incubated with JC-1 (working concentration of 10 μg ml⁻¹) in warm D-PBS containing 10 μg ml⁻¹ DAPI for 10 min. Then cells were washed three times with PBS and imaged with a confocal microscope (Zeiss LSM 710). For JC-1 monomers, 488-nm excitation wavelength was used and fluorescence signals at 525±15 nm were collected as the green channel. For JC-1 aggregates, 546-nm excitation wavelength was used and fluorescence signals at 600±15 nm were collected as the red channel.

### Example 12

Subcellular localization: Subcellular localization of CDN was determined by analyzing the colocalization of CDM signal with subcellular organelles labeled by commercially available trackers. Briefly, cells were seeded (2×10⁴ per well) in a Lab-Tek II chamber (NUNC) and cultured in a humidified atmosphere of 5% CO₂ and 95% air at 37° C. overnight before the experiments. For mitochondria, cells were co-incubated with MitoTracker (Thermo Fisher, M7514) at a working concentration of 200 nM and CDN (CDM, 1 μM) for 30 min. For endoplasmic reticulum, cells were pre-incubated with CDN (CDM, 0.1 μM) for 4 h. Cells were then washed with HBSS three times and stained with ER-tracker Green (Thermo Fisher, E34251) for 30 min. For the early endosome, cells were preincubated with CellLight Early Endosomes-GFP (Thermo Fisher, C10586) overnight, then incubated with CDN for 1 h before imaging. Nucleus staining was done in all experiments with Hoechst 33342 (10 μg ml⁻¹; Thermo Fisher, cat. no. 62249). The confocal microscopic imaging settings were 488 nm excitation/525±15 nm emission for the green channel and 633 nm excitation/650-730 nm emission for the red channel. The correlation was drawn out of three different views of cells imaged with a ×20 lens, with approximately 150 cells per view. Pearson r value was calculated using ImageJ with Costes threshold regression.

### Example 13

COX activity assay: COX activity was measured by Cytochrome C Oxidase Assay Kit (Abcam, ab239711). Briefly, 2×10⁷ cells were incubated with CDN or control agents. At 24 h after incubation, cells were collected and washed three times with cold PBS. The cell pallets were processed with Mitochondria Isolation Kit for Cultured Cells (Thermo Fisher, cat. no. 89874) to obtain mitochondrial extraction. After protein quantification, 5 μg of total purified mitochondrial protein was used for COX activity measurement. The activity of the enzyme was determined by measuring the oxidation of reduced cytochrome c as an absorbance decrease at 550 nm. The rate of the enzyme reaction was calculated in the linear range (n=3 for each treatment condition).

### Example 14

OCR and ECAR measurement. OCR and ECAR were measured by a Seahorse Bioscience instrument (XF96, Agilent). Briefly, on the day following MDA-MB-231 or MDA-MB-468 cell seeding, cells were incubated with CDN or control agents for 24 h (chelator concentration, 1 μM). On the day of measurement, cells were washed three times with D-PBS, changed into fresh medium without phenol red and bicarbonate (Seahorse base medium, cat. no. 103336-100) and equilibrated for 30 min in a 37° C. incubator lacking CO₂. Oxygen concentration and extracellular acidification in media were measured at basal conditions and after sequential addition of oligomycin (1 μM, introduced after 28 min), FCCP (1 μM, introduced after 54 min) and rotenone/antimycin A (0.5 μM, introduced after 80 min) (n=8 per condition for OCR and ECAR for each cell line). For acute OCR inhibition titration assay and copper remedy assay, on the day following cell seeding, cells were incubated with CDN at various CDM concentrations for 1 h or with 1 μM Cu(II). Cells were then changed into Seahorse base medium and equilibrated for 30 min in a 37° C. incubator lacking CO₂. Oxygen concentration and extracellular acidification in media were measured at basal conditions and after sequential addition of oligomycin (1 μM, introduced after 28 min), FCCP (1 μM, introduced after 54 min) and rotenone/antimycin A (0.5 μM, introduced after 80 min) (n=6 per condition for OCR and ECAR for each cell line).

### Example 14

Cellular ATP measurement: ATP levels after CDN or control agent treatment were determined by Adenosine 5-triphosphate Bioluminescent Somatic Cell Assay Kit (Sigma-Aldrich, FLASC). Briefly, 10⁶ cells were seeded in six-well plates 24 h before the treatment. Other than the control group, cells were incubated with CDN, SPN, TPA or ATN224 at concentrations indicated in the figure legends for 24 h in complete medium or medium without FBS supplement. For reactive oxygen species (ROS) scavenger experiments, various concentrations of MitoTEMPO were added to the medium for coincubation. Cells were then trypsinized and resuspended in ultrapure water for assay. The luminescence signals were measured by a Turner Biosystems 20/20n luminometer. The ATP concentration of each group was determined based on the ATP standard assay (n=3 per condition ATP production measurement for each cell line).

### Example 15

Glucose uptake and lactic acid secretion. Glucose uptakes from the culture medium and lactic acid secretion were measured by assay kits according to the manufacturer protocols. Briefly, cells were seeded in six-well plates (10⁶ per well) 24 h before the treatment and cultured in a humidified atmosphere of 5% CO₂ and 95% air at 37° C. Then cells were incubated with 2 ml of fresh medium containing CDN or control agents for 24 h. The same volume of medium in wells without cell seeding was used as baseline (same culturing conditions). The glucose level in the medium was measured by Glucose Colorimetric Assay Kit (Cayman Chemical, 10009582), and calculated as the decrease in medium glucose level after incubation. The lactic acid secretion into the medium was measured by Glycolysis Cell-Based Assay Kit (Cayman Chemical, 600450), and calculated as the increase of lactate in the medium after incubation (n=3 per condition for glucose uptake and lactate secretion, respectively).

### Example 16

In vitro labeling using ¹³C6-glucose or ¹³C5,¹⁵N2-glutamine: MDA-MB-231 cells were plated into 10-cm dishes and cultured at 37° C. with 5% CO₂ before treatment. On the day of treatment, for the ¹³C6-glucose-labeled groups, normal medium was replaced with medium containing 2 g l⁻¹-labeled ¹³C6-glucose (Sigma, cat. no. 660663) instead of ¹²C6-glucose. For the ¹³C5,¹⁵N2-glutamine-labeled groups, normal medium was replaced with medium containing 0.3 g l⁻¹-labeled ¹³C5,¹⁵N2-glutamine (Sigma, cat. no. 607983) instead of ¹²C5,¹⁴N2-glutamine. Half of the dishes of cells grown in non-labeled control media, half of the dishes of cells grown in ¹³C6-glucose-labeled media and half of the dishes of cells grown in ¹³C5,¹⁵N2-glutamine-labeled media were treated with CDN (CDM, 1 μM), while the other halves were treated with control (n=5 per group). Cells were collected 24 h after treatment.

### Example 17

Metabolomics analysis of labeled in vitro samples: The cells were subjected to metabolite extraction using the protocol as previously described (Nguyen et al., (2019) Cell Reports 27: 491-501; Udupa et al., (2019) Proteomics 19:1800451; Elgogary et al., (2016) Proc. Natl Acad. Sci. U.S.A. 113: E5328-E5336). The lyophilized aqueous-phase metabolite samples were resuspended in 50% (vol/vol) acetonitrile diluted with mass-spectrometry-grade water, while the dried organic-phase metabolite samples were resuspended in 2:1 (vol/vol) chloroform/methanol. Metabolomics data from the samples were acquired using a Thermo Scientific Q Exactive Orbitrap Mass Spectrometer Plus with a Vanquish UPLC system. The Vanquish UPLC auto-sampler systems were used to uptake 2 μl of each sample and were maintained at 4° C. Reverse-phase chromatography used 0.1% formic acid in mass-spectrometry-grade water as the mobile aqueous phase and 0.1% formic acid in 98% acetonitrile as the mobile organic phase. The total run time for each sample was 13 min. A Discovery HS F5 HPLC Column with 3-μm particle size, 15-cm length, and 2.1-mm internal diameter (Sigma) and a compatible guard column (Sigma) were used and were maintained at 35° C. Data were analyzed using Thermo Fisher Scientific Compound discoverer, Xcalibur and TraceFinder software. The raw intensities were normalized based on protein concentration and cell weight of each sample to get the final normalized intensities.

### Example 18

Metabolomics analysis of in vivo samples: MDA-MB-468 tumors in control and CDN treatment groups were subjected to metabolite extraction as described in Example 17 (n=5 for each group). The lyophilized samples were resuspended in 50% (vol/vol) acetonitrile diluted with mass-spectrometry-grade water. Data were analyzed using Thermo Fisher Scientific Compound discoverer, Xcalibur and TraceFinder software. The raw intensities were normalized based on protein concentration and tumor weight of each sample to get the final normalized intensities.

### Example 19

mRNA sequencing: A total of 107 MDA-MB-231 cells in 75-cm² flasks were incubated with CDN (CDM, 1 μM) for 15 h or 24 h. Then nontreated control and CDN-treated cells were collected and resuspended in Trizol (Life Technology). The RNA extraction, sample quality control, library preparation and sequencing (6 G raw data per sample) were performed by Novogene Corporation. For the data analysis, each condition had an average of 25.5 million 150-base-pair-long paired-end reads. Fastqc (v.0.11.2) was used for sequencing quality assessment (Multiqc (v.1.5) was used to aggregate results into a single report). Reads were then aligned to GRCh38(hg38) genome using STAR v.2.5.3a with splice junctions being defined in a GTF file (obtained from GRCh38). An average of 89% of reads were aligned to the reference genome. Expression at the gene level was determined by calculating reads per kilobase per million aligned reads as well as raw count using RSEM (v.1.2.30).

### Example 20

SOD activity and oxidative stress: (1) SOD activity measurement: 1.5×10⁶ cells in 25-cm² flasks were incubated with CDN or control agents for 24 h. After treatment, cells were gently washed twice with D-PBS, followed by trypsinization and centrifugation at 1,000 relative centrifugal force (rcf) for 10 min at 4° C. Then 500 μl of cold HEPES buffer was added to the pellet, followed by sonication with a probe sonicator for 10 s in an ice bath (six RWS, twice). After centrifugation at 1,500 rcf for 5 min at 4° C., the supernatant was removed for assay and stored at −80° C. before the test. For sample measurement, 100 μl of each sample was centrifuged at 10,000 rcf for 15 min at 4° C. The supernatant was collected as the cytosolic content and measured for the protein concentration with BCA assay (Thermo Scientific, cat. no. 23250). The sample SOD activity was measured with Superoxide Dismutase Assay Kit (Cayman Chemical, 706002) according to the provided protocol (n=3 per condition).
Superoxide measurement: the superoxide level after CDN or control agent treatment was measured by a chemiluminescent sensor for superoxide. Briefly, after 24 h of pretreatment with CDN or control agents, cell suspension (10⁶ cells per ml in PBS) was collected, mixed with 200 μl of CLA (TCI America, cat. no. A5307) in HBSS solution (working concentration of 3.6 μg ml-1) and incubated at 37° C. for 3 min. The chemiluminescence signals were measured by a Turner Biosystems 20/20n luminometer (n=3 per condition).
DNA damage and lipid peroxidation caused by oxidative stress: the DNA damage and lipid peroxidation in cells after CDN or control agent treatment for 24 h were determined by immunofluorescent staining. DNA damage was detected by anti-γH2A.X (phosphor S139) antibody (Abcam, ab11174), and lipid peroxidation was detected by anti-4-hydroxynonenal antibody (Abcam, ab46545).

### Example 21

Animal models: All mice used in the animal experiments were 6-8-week-old female mice purchased from Charles River. For animal models used in labile copper bioluminescence imaging experiments, 2×10⁶ MDA-MB-231luc cells were inoculated into the fourth mammary fat pad of NSG mice to generate orthotopic models. For animal models used in MAL3 bioluminescence imaging experiments, 3×10⁶ MDA-MB-231luc cells were inoculated into the fourth mammary fat pad of Swiss nu/nu mice to generate orthotopic models. For treatment efficacy and survival studies, 5×10⁵ 4T1 cells were inoculated into the fourth mammary fat pad of Blab/c mice, or 2×10⁶ MDA-MB-231 or MDA-MB-468 cells were inoculated into the fourth mammary fat pad of nude mice to generate orthotopic models.

### Example 22

Copper depleting dynamics and tumor retention of CDN in vivo: For the copper depleting dynamics study, the labile copper levels in tumor were determined by CCL-1 imaging. MDA-MB-231luc tumor-bearing mice were intraperitoneally injected with freshly prepared CCL-1 in D-PBS solution (6 mg kg⁻¹) before and at each time point after CDN injection (i.v., CDM, 1.35 mg kg⁻¹). At 30 min after CCL-1 administration, mice were imaged with IVIS Spectrum for luminescence signal (n=3). For the tumor retention- and copper depletion-monitoring study, MDA-MB-231 tumor-bearing mice were intravenously injected with CDN (CDM, 1.35 mg kg⁻¹). The fluorescence signal from CDM and the photoacoustic signal from SPN were acquired before and at days 1, 2, 3, 5, 7, 10 and 14 after CDN administration. The CDM fluorescence was acquired with IVIS Spectrum with an excitation at 640 nm and an emission filter at 740 nm. The photoacoustic signal from SPN was acquired with LAZR VisualSonics. Three-dimensional photoacoustic and ultrasound images were recorded. Laser pulses at wavelength 1,064 nm were routed directly from the pump laser (Q switch Nd:YAG laser) of an optical parametric oscillator system. The laser pulses were coupled to an optical fiber bundle that was integrated on the ultrasound transducer. The averaged output fluence of the laser was adjusted by neutral density filters to 60±3 mJ cm-2 (7 ns). To record photoacoustic and ultrasound images, an ultrasound/photoacoustic micro-imaging system (LAZR, VisualSonics) was used with a 40-MHz array ultrasound transducer (LZ550, VisualSonics). A volume of 14×15×15 mm³ was mechanically scanned with a step size of 63 μm. Given the imaging parameters, each three-dimensional scan required 240 frames.

### Example 23

Mitochondrial membrane potential imaging in vivo: A total of 30 Swiss nu/nu (female, 8 weeks old) mice were injected with a suspension of MDA-MB-231luc cells (3×10⁶ cells per mouse). When half of the animals developed tumors of about 150-300 mm³ (this reflects approximately 50% take rate for this cell line), mice were split into three groups (five mice per group) and imaged. All of the mice were imaged with mitochondrial membrane potential (ΔΨm)-specific bioluminescent MAL3 probe to assess the basal level of ΔΨm in the tumors 4 d before CDN or control agent administration.

The MAL3 probe consisted of two reagents that result in light production proportional to the level of ΔΨm (TPP-CL2 and azido-TPP1)22. In a typical ΔΨm imaging experiment, mice received i.v. injection of 100 μl of 700 μM TPP-CL2 solution in the vehicle comprising 0.1% fatty acid-free BSA in PBS. After 20 h, the animals received intraperitoneal injection of azido-TPP1 solution (100 μl, 7 mM in PBS) followed by continuous imaging during 50 min using IVIS Spectrum (PerkinElmer) with the following settings: exposure 3 min, binning 16, aperture F1, field of view D. To take into account the heterogeneity of tumor sizes, all of the mice received subsequent intraperitoneal injection of a large dose of luciferin potassium salt solution in PBS (100 μl, 33 mM) and were imaged for another 10 min using the following settings: exposure auto, binning 8, aperture F1, field of view D.

Next, the mice were allowed to rest for 3 d to assure clearance of the imaging probes. On day 0 of the experiment, mice in each group received i.v. injection of 150 μl of PBS, SPN solution or CDN (CDM, 1.35 mg kg⁻¹) solution. ΔΨm in tumor xenografts was monitored using MAL3 probe on days 2 and 8. Similar to day −4, the mice were imaged with luciferin immediately after being imaged with MAL3 probe to account for differences in tumor size.

The total photon flux from each mouse was quantified by calculating the area under the kinetic curve resulting after MAL3 and luciferin injections. The total photon flux from the MAL3 probe was normalized to the total photon flux resulting from the injection of luciferin for each animal to take into account differences in tumor size. Resulting normalized values on day −4 (before injection of nanoparticles and corresponding controls), day 2 (2 d after the injection of reagents) and day 8 (8 d after the reagent injection) for each group were plotted.

### Example 24

Tissue distribution: To determine the ex vivo tissue distribution of CDN, MDA-MB-231 tumor-bearing mice (n=6) were intravenously injected with fCDN (CDM, 1.35 mg kg⁻¹). At 24 h after injection, mice were whole-body imaged for CDM and fSPN fluorescence and then killed to collect major organs for ex vivo imaging, including tumor, mammary fat pad, heart, lung, liver, spleen, adrenal, kidney, stomach, small intestine, large intestine, ovary and uterus. The excitation wavelength was 500 nm; the emission wavelength for green fluorescence from fSPN was 540 nm, and for red fluorescence from CDM was 740 nm. After fluorescence imaging, tumor and liver tissues were embedded in Tissue-Tek O.C.T. (optimal cutting temperature) gel and the frozen tissue blocks were sliced for microscopic imaging. Confocal microscopic imaging settings for tissue slices were excitation wavelength at 488 nm, and emission wavelengths from 500 to 560 nm for green fluorescence and from 610 to 733 nm for red fluorescence.

### Example 25

Therapeutic effect of CDN: (1) Copper level after treatment: MDA-MB-231luc tumor-bearing mice were randomly divided into four groups (n=5 per group): control, SPN (90 mg kg⁻¹), TPA (1.35 mg kg⁻¹) and CDN (90 mg kg⁻¹ of SPN+1.35 mg kg⁻¹ of CDM). For the treatment group, mice were intravenously injected with CDN or control agents every 3 d, with five doses in total. Tumor volume was continuously monitored over the treatment plan. At day 25 after initial treatment, mice were subjected to d-luciferin bioluminescence imaging (150 mg kg⁻¹) and CCL-1 luminescence imaging (6 mg kg⁻¹).

(2) Long-term survival study: MDA-MB-231 tumor-bearing mice were randomly divided into six groups (n=12 per group): control, SPN, TPA, ATN224, CDM and CDN groups. TPA, CDM and CDN groups received an i.v. injection of the corresponding agent at the same chelator concentration of 1.35 mg kg⁻¹ weekly for seven doses in total. The SPN group received an i.v. injection of the same amount of SPN as in the CDN group weekly for seven doses in total. The ATN224 group received a daily oral gavage of ATN224 at a dose of 0.7 mg kg⁻¹ d⁻¹ for 42 d at maximum. The treatment plan was initiated when the average tumor size reached 50 mm³ and the endpoint of the survival study was primary tumor volume over 500 mm³ and/or other signs identified by the veterinarian. The tumor volumes were continuously monitored over the treatment plan.

Therapeutic efficacy in the 4T1 model: 4T1 tumor-bearing mice were randomly divided into three groups (n=5 per group): control, TPA and CDN groups. TPA and CDN groups received an i.v. injection of the corresponding agent at the same chelator concentration of 1.35 mg kg⁻¹ weekly for five doses in total. The treatment plan was initiated when the average tumor size reached 50 mm³ and the endpoint was the primary tumor volume over 500 mm³. All surviving animals were euthanized at day 32 after the initial treatment to compare the tumor sizes after treatment.
Therapeutic efficacy in the MDA-MB-468 model and in vivo metabolomics study: MDA-MB-468 tumor-bearing mice were randomly divided into five groups (n=5 per group): control, SPN, TPA, ATN224 and CDN groups. TPA and CDN groups received an i.v. injection of the corresponding agent at the same chelator concentration of 1.35 mg kg⁻¹ weekly for seven doses in total. The SPN group received an i.v. injection of the same amount of SPN as in the CDN group weekly for seven doses in total. The ATN224 group received a daily oral gavage of ATN224 at a dose of 0.7 mg kg⁻¹ d⁻¹ for 42 d at maximum. The treatment plan was initiated when the average tumor size reached 50 mm³. The tumor volumes were continuously monitored over the treatment plan. At 3 d after the last injection, animals were killed. Tumors were collected, weighed and snap-frozen for metabolite extraction and analysis.

### Example 26

Acute toxicity and cumulative toxicity of CDN: For the acute toxicity study, healthy female Balb/c mice were intravenously injected with saline or CDN (CDM, 100 mg kg⁻¹) and monitored for 2 weeks (n=3 per group). Mice were observed for behavioral changes, with body weight monitored over 2 weeks, before being killed for hematological analysis, Mammalian Liver Profile tests, and tissue histology analysis. Tissues to examine included heart, lungs, liver, kidneys and spleen.

For the cumulative toxicity study, healthy female Balb/c mice were intravenously injected with saline or CDN (CDM, 1.35 mg kg⁻¹) weekly for a total of seven doses (n=5 per group). Mice were observed for behavioral changes, and body weight was monitored over the treatment course. Mice then were killed at day 45 after initial treatment for hematological analysis, Mammalian Liver Profile tests and tissue histology analysis. Tissues to examine included heart, lungs, liver, kidneys, and spleen.

### Example 27

Statistical analysis: Unpaired t-test was performed to compare two groups (two-tailed). Kruskal-Wallis tests were performed for multiple-group comparisons and Wilcoxon rank sum test was conducted for post hoc pair comparisons. For the survival study, log rank tests were conducted for multiple groups in overall survival.

### Example 28

CDM synthesis: CDM was prepared according to reported procedures with some modifications (Kiyose et al., (2006) J. Am. Chem. Soc. 128: 6548-6549).

(1) Synthesis of compound 3: N-Boc-Ethylenediamine (0.8 g, 5 mmol), Na2CO3 (2.4 g, 22.6 mmol), and 2-chloromethylpyridine hydrochloride (1.8 g, 10.9 mmol) were dissolved in methanol and refluxed under argon for 48 h. Then the solvent was evaporated. The residue was dissolved in 1N NaOH solution and extracted by dichloromethane (DCM). The organic phase was dried by anhydrous sodium sulfate and evaporated to give a yellow liquid that was purified by chromatography (Al2O3, DCM/MeOH) to afford 2. Compound 2 (519 mg, 1.508 mmol) was dissolved in DCM to which 3 mL of trifluoroacetic acid was added dropwise on an ice bath. The mixture was stirred under room temperature for 3 h before 1 N NaOH was added to neutralize the solution. The organic phase was dried by anhydrous sodium sulfate and evaporated to get a yellow liquid 3 (335 mg, yield 92%). ¹H NMR (300 MHz, CDCl3): δ 1.92 (s, 2H), 2.67 (t, 2H, J=5.7 Hz), 2.80 (t, 2H, J=5.7 Hz), 3.85 (s, 4H), 7.12 (m, 2H), 7.49 (d, 2H, J=7.7 Hz), 7.63 (td, 2H, J=7.7 Hz, 1.83 Hz), 8.52 (dd, 2H, J=4.9 Hz, 0.8 Hz). 13C NMR (75 MHz, CDCl3): δ 39.1, 56.7, 60.1, 121.5, 122.5, 135.9, 148.5, 159.1. MS (ES⁺): m/z calculated for C14H18N4+: calculated 243.1; found 243.2 (M+H)⁺.

(2) Synthesis of CDM: The last step in Scheme 1 followed reported procedures with slight modifications (Fanagan et al., (1997) Bioconjugate Chem. 8: 751-756). Briefly, IR780 (325 mg, 0.509 mmol), compound 3 (370 mg, 1.528 mmol), and anhydrous K2CO3 (71 mg, 0.51 mmol) were dissolved in anhydrous DMF. The solution was heated to 80° C. and stirred for 5 h. The solvent was evaporated under reduced pressure and washed with water and brine for three times. After adding DCM, the organic layer was extracted and dried by anhydrous sodium sulfate. The crude product was purified by silica column chromatography (DCM/MeOH=50:1 to 20:1) to give final product CDM (167 mg, yield 39%). ¹H NMR (400 MHz, CDCl3): δ 8.44-8.42 (m, J=8.0 Hz, 2H), 7.68-7.61 (m, J=28.0 Hz, 4H), 7.44-7.42 (d, J=8.0 Hz, 2H), 7.26-7.18 (m, J=32.0 Hz, 4H), 7.14-7.11 (m, J=12.0 Hz, 2H), 7.02-6.98 (t, J=16.0 Hz, 2H), 6.82-6.80 (d, J=8.0 Hz, 2H), 5.58-5.55 (d, J=12.0 Hz, 2H), 4.02 (s, 4H), 3.82-3.73 (m, J=32.0 Hz, 6H), 3.15-3.13 (m, J=8.0 Hz, 2H), 2.52-2.49 (m, J=12.0 Hz, 4H), 1.82-1.78 (m, J=16.0 Hz, 6H), 1.56 (s, 10H), 1.24 (s, 2H), 1.03-0.99 (m, J=16.0 Hz, 6H). ¹³C NMR (100 MHz, CDCl3): δ 166.49, 158.74, 149.29, 143.54, 139.97, 136.91, 128.26, 123.66, 122.52, 122.45, 122.30, 122.10, 120.16, 108.39, 93.68, 60.08, 53.81, 47.82, 47.51, 44.76, 28.93, 26.30, 21.44, 20.19, 11.98. MS: m/z calculated for C50H51N6+: 745.5; found: 745.5. 1H NMR

### Example 29

### Semiconducting Polymer Synthesis:

(1) Synthesis of N-(2-octyldodecyl)thiophen-3-amine (6): 3-Bromothiophene (13.04 g, 80 mmol), 2-octyldodecan-1-amine (29.76 g, 100 mmol), potassium phosphate tribasic (21.23 g, 100 mmol), and copper (1) iodide (1.53 g, 8 mmol) were added into 90 mL of dimethyl aminoethanol in a 250 mL round-bottom flask, which was vacuumed and purged with argon three times, and then stirred at 90° C. for 48 h. After cooling down to room temperature, the mixture was filtered, and solvent was removed under reduced pressure. The crude product was purified by column chromatography on silica gel using ethyl acetate/hexane (v/v 1:10) as the eluent to afford 6 as a liquid (16.3 g, yield 43%). 1H NMR (400 MHz, CDCl3, 25° C.) δ (ppm): 7.14 (d, 1H), 6.62 (d, 1H), 5.91 (d, 1H), 3.59 (br, 1H), 2.96 (d, 2H), 1.59 (br, 1H), 1.06-1.44 (m, 32H), 0.88 (m, 6H). 13C NMR (100 MHz, CDCl3, 25° C.) δ (ppm): 149.18, 124.95, 119.93, 94.79, 49.91, 37.84, 32.23, 31.92, 30.08, 29.66, 29.35, 26.80, 22.69, 14.11.

(2) Synthesis of 4-(2-octyldodecyl)-4H-thieno[3,2-b]pyrrole-5,6-dione (8): To a solution of oxalyl dichloride (6.35 g, 50 mmol) in 40 mL of dichloromethane was added dropwise a solution of compound 6 (15.19 g, 40 mmol) and 70 mL of dichloromethane at 0° C. After 30 min, 13 mL of triethylamine in 15 mL of dichloromethane was added dropwise, and the mixture was warmed to room temperature and stirred overnight. Then the solvents were removed under reduced pressure and the residue was purified by column chromatography on silica gel using dichloromethane/hexane (v/v 1:2) as the eluent to afford 4-(2-octyldodecyl)-4H-thieno[3,2-b]pyrrole-5,6-dione (8) as a red oil (7.8 g, yield 45%). ¹H NMR (400 MHz, CDCl3, 25° C.) δ (ppm): 7.98 (d, 1H), 6.76 (d, 1H), 3.52 (d, 2H), 1.30 (br, 1H), 1.12-1.44 (m, 32H), 0.88 (m, 6H). ¹³C NMR (100 MHz, CDCl3, 25° C.) δ (ppm): 172.97, 165.53, 161.75, 143.77, 113.15, 111.04, 46.47, 36.98, 31.89, 31.84, 31.40, 29.89, 29.59, 29.53, 29.48, 29.31, 29.25, 26.36, 22.66, 22.64, 14.09, 14.08.

(3) Synthesis of (e)-4,4′-bis(2-octyldodecyl)-[6,6′-bithieno[3,2-b]pyrrolylidene]-5,5′(4H,4′H)-dione (10). Lawesson's reagent (3.24 g, 8 mmol) was added to a solution of compound 8 (6.51 g, 15 mmol) and 40 mL of toluene, which was vacuumed and purged with argon three times, and the mixture was stirred at 75° C. for 3 h. Then the solvent was removed under reduced pressure, and the residue was purified by column chromatography on silica gel using dichloromethane/hexane (v/v 1:4) as the eluent to afford 10 as a dark purple solid (4.72 g, yield 38%). ¹H NMR (400 MHz, CDCl3, 25° C.) δ (ppm): 7.51 (d, 2H), 6.78 (d, 2H), 3.69 (d, 4H), 1.89 (br, 2H), 1.05-1.43 (m, 64H), 0.87 (m, 12H). ¹³C NMR (100 MHz, CDCl3, 25° C.) δ (ppm): 171.30, 151.54, 134.17, 121.05, 114.25, 111.32, 46.16, 37.15, 31.91, 31.87, 31.49, 29.95, 29.61, 29.56, 29.51, 29.33, 29.27, 26.42, 22.68, 22.65, 14.11, 14.10.

(4) Synthesis of 2,2′-dibromo-4,4′-bis(2-octyldodecyl)-[6,6′bithieno[3,2,b]pyrrolylidene]-5,5′(4H,4′H)-dione (11): N-bromosuccinimide (2.14 g, 12 mmol) was added to compound 10 (4.18 g, 5 mmol) in 50 mL of THF at 0° C. in the absent of light. After stirred for about 2 h, the reaction mixture was poured into water and extracted with hexane. The organic layer was dried with anhydrous Na2SO4 and the solvent was evaporated under reduced pressure. The residue was purified by column chromatography on silica gel using dichloromethane/hexane (v/v 1:4) as the eluent to afford 2,2′-dibromo-4,4′-bis(2-octyldodecyl)-[6,6′bithieno[3,2,b]pyrrolylidene]-5,5′(4H,4′H)-dione as a dark blue solid (3.55 g, yield 71%). ¹H NMR (400 MHz, CDCl3, 25° C.) δ (ppm): 6.81 (s, 2H), 3.61 (d, 4H), 1.83 (m, 2H), 1.07-1.41 (m, 64H), 0.87 (m, 12H). ¹³C NMR (100 MHz, CDCl3, 25° C.) δ (ppm): 170.32, 150.16, 123.06, 119.62, 114.94, 114.68, 46.18, 37.19, 31.92, 31.88, 31.41, 29.95, 29.63, 29.57, 29.51, 29.35, 29.29, 26.36, 22.69, 22.67, 14.11, 14.10.

(5) Synthesis of SP: Compound 11 (397 mg, 0.4 mmol), hexamethyldistannane (131 mg, 0.4 mmol) and dry chlorobenzene (10 mL) were added to a 50 mL of degassed and dried Schlenk tube. The solution was purged with argon for 30 min, followed by the addition of Pd2(dba)3 (20 mg), and then vacuumed and charged with argon three times. The reaction mixture was stirred vigorously at reflux for 72 h under argon atmosphere. Afterwards, the solution was poured into 300 mL of methanol and stirred for 4 h, filtered and then extracted on a Soxhlet's extractor with methanol, acetone, hexane, and chloroform successively. The final chloroform solution was concentrated, and poured into methanol, and the polymer was collected by filtration and dried under reduced pressure at room temperature for 24 h as a black solid (238 mg, yield 71%). (C52H86N2O2S2)n (835.39)n: Calcd. C, 74.76; H, 10.38; N, 3.35; 0, 3.83; S, 7.68. Found C, 74.78; H, 10.36; N, 3.33; 0, 3.78; S, 7.75. GPC Mn=33.0 kDa, Mw=76.5 kDa; PDI=2.32.

### Example 30

mRNA Sequencing Analysis:

(1) Heatmap scale (Gaujoux & Seoighe (2010) BMC Bioinformatics 11: 367), after hierarchical clustering using Median Clustering Or Weighted Pair Group Method With Centroid Averaging (WPGMC) method together with “Spearman” correlation (square of Euclidean distance) method for distance measurement on the log-transformed gene expression table, the values are further scaled in the row direction, which centers and standardizes each row separately to row Z-score.

(2) Principal-component analysis (PCA): Principal-component analysis of the populations (using around 46.7% (12328 genes in total 26475 genes) transcripts of all genes after minimal pre-filtering to keep only rows which have at least 10 read/raw counts in total). Numbers indicate frequency of transcripts described by each principal component. PCA was performed to extract the main information from the DESeq2 transformed (r log) data set so that each successive axis was ordered by decreasing order of variance, which plot helps to visualize the batch effects and overall effect of experimental covariates. (Love et al., (2014) Genome Biol. 15: 550)

(3) Distance matrix: sample-to-sample distance matrix (name of the distance matrix) indicating degree of similarity between selected cell populations calculated using (the screened transcripts (46.7%) of all genes); 12328 genes in plot indicate sample-to-sample distance. Based on the data set after differential expression analysis performed using DESeq2 measuring the effect of condition, the Euclidean distances between the samples as calculated from the r log transformation were calculated and plotted as heatmap. (Love et al., (2014) Genome Biol. 15: 550)

(4) Heatmap of selected genes (genes are ordered by fold change): The selected genes were extracted from the matrix generating the general heatmap, hence the heatmaps were generated according to the normalized data set scaled in the row direction. (Renaud Gaujoux, 2014. Aheatmap: a Powerful Annotated Heatmap Engine. Package NMF—Version 0.22)

(5) The raw count table for the three conditions (Control, 15 h and 24 h) was obtained from RSEM analysis on STAR mapping. Then the normalization was performed across the samples based on the size factor (estimated SizeFactors function). The median of the ratios of observed counts was used in the following formula (Genome Biology 2010, 11, R106):

$$\hat{s} j = median ( i ) \frac{{k}_{ij}}{{\{ {n}_{v = 1}^{m} {k}_{iv} \}}^{1 / m}} \qquad ( 1 )$$

where the denominator of this expression is as a pseudo-reference sample obtained by taking the geometric mean across samples and the size factor estimate ŝj is computed as the median of the ratios of the j-th sample's counts to those of the pseudo-reference.

Based on the normalized count table, the correlations between two conditions were calculated and plotted. Here, we use two comparison correlation: Ctrl vs 15 hrs and Ctrl vs 24 hrs. The Fold Change (15 h/Control and 24 h/Control) of 1.5 was set as cutoff threshold, and the genes with Fold Change above 1.5 are upregulated genes and the ones with Fold Change below −1.5 are downregulated genes. Then the overlapped genes between the two comparisons were collected for upregulated and downregulated gene set separately and generate the new upregulated and downregulated gene lists. These genes lists were then fed to DAVID Functional Annotation Tool to perform GO-Term analysis. The extracted term chart was exported as the results.

What is claimed:

1. A nanoparticle comprising a self-reporting copper-depleting moiety (CDM) and a matrix, wherein the matrix comprises a semi-conducting polymer and a phospholipid-polyethylene glycol (PEG), wherein the CDM is embedded in or on the matrix.

2. The nanoparticle of claim 1, wherein the copper-depleting moiety (CDM) is linked to a fluorescent moiety.

3. The nanoparticle of claim 1, wherein the copper-depleting moiety (CDM) is N,N-bis(2-pyridinylmethyl)-1,2-ethanediamine.

4. The nanoparticle of claim 2, wherein the fluorescent moiety is a tricarbocyanine.

5. The nanoparticle of claim 2, wherein the linkage of the CDM to the fluorescent moiety is configured to allow the CDM, when bound to a copper ion, to quench a fluorescence emission from the fluorescent moiety.

6. The nanoparticle of claim 1, wherein the semi-conducting polymer is a photoacoustic semi-conducting polymer.

7. A method of reducing the amount of free copper ions in the mitochondria of a target cell or population of target cells, the method comprising the step of delivering a nanoparticle to the interior of a target cell, wherein the nanoparticle comprises (a) a matrix comprising a semi-conducting polymer and a phospholipid-polyethylene glycol (PEG); and (b) a self-reporting copper chelator (CDM) embedded in or on the matrix, whereby copper ions in the mitochondria of the recipient cell chelate to the nanoparticle, thereby reducing the level of free copper in the mitochondria.

8. The method of claim 7, wherein the copper-depleting moiety (CDM) is linked to a fluorescent moiety, wherein the linkage of the CDM to the fluorescent moiety is configured to allow the CDM, when bound to a copper ion, to quench a fluorescence emission from the fluorescent moiety.

9. The method of claim 7, wherein the copper-depleting moiety (CDM) is N,N-bis(2-pyridinylmethyl)-1,2-ethanediamine.

10. The method of claim 8, wherein the fluorescent moiety is a tricarbocyanine.

11. The method of claim 7, wherein the semi-conducting polymer is a photoacoustic semi-conducting polymer.

12. The method of claim 7, wherein the target cell is a cancer cell and the population of target cells is in a tumor.

13. The method of claim 12, wherein the target cell or the population of target cells is in an animal or human subject tumor.

14. The method of claim 7, further comprising the step of detecting a change in an optical signal generated from the nanoparticle by chelation of copper ions to the nanoparticle.

15. The method of claim 7, further comprising the step of detecting a photoacoustic signal from the nanoparticle and determining the location of the nanoparticle within the animal or human subject.

16. A method of reducing at least one of the proliferation and the viability of a cancer cell by administering a self-reporting copper-depleting moiety (CDM) and a matrix, wherein the matrix comprises a semi-conducting polymer and a phospholipid-polyethylene glycol (PEG), wherein the CDM is embedded in or on the matrix, wherein copper ions in the mitochondria of the recipient cell are chelated by the CDM, thereby reducing the level of free copper in the mitochondria and reducing at least one of the proliferation and the viability of a cancer cell in the patient.

17. The method of claim 16, wherein the copper-depleting moiety (CDM) is linked to a fluorescent moiety, wherein the linkage of the CDM to the fluorescent moiety is configured to allow the CDM, when bound to a copper ion, to quench a fluorescence emission from the fluorescent moiety, wherein the copper-depleting moiety (CDM) is N,N-bis(2-pyridinylmethyl)-1,2-ethanediamine, the fluorescent moiety is a tricarbocyanine and the semi-conducting polymer is a photoacoustic semi-conducting polymer.

18. The method of claim 16, wherein the cancer is Triple Negative Breast Cancer (TNBC).

19. The method of claim 16, further comprising the step of detecting at least one of an optical signal from the nanoparticle generated after chelation of copper ions to the nanoparticle and detecting the presence of the cancer in the patient by detecting a change in the optical signal from the nanoparticle generated by chelation of copper ions to the nanoparticle and determining the location of the cancer in the patient.

20. The method of claim 16, further comprising adjusting the level of a dose of a therapeutic agent administered to the patient in need thereof, whereby the change of the intensity of the optical signal indicates the level of the reduction of the proliferation or viability of the cancer cell caused by the therapeutic agent
